(19) United States
(12) Patent Application Publication
(10) Pub. No.: US 2014/0039803 A1
(43) Pub. Date: Feb. 6, 2014

(54) Method for Rapid Identification of Drug Targets and Drug Mechanisms of Action in Human Cells

(71) Applicants: Olivier Elemento, New York, NY (US); Tarun M. Kapoor, New York, NY (US); Sarah A. Wacker, New York, NY (US)

(72) Inventors: Olivier Elemento, New York, NY (US); Tarun M. Kapoor, New York, NY (US); Sarah A. Wacker, New York, NY (US)

(73) Assignees: THE ROCKEFELLER UNIVERSITY, New York, NY (US); CORNELL UNIVERSITY, Ithaca, NY (US)

(21) Appl. No.: 14/003,065

(22) PCT Filed: Mar. 2, 2012
(86) PCT No.: PCT/US12/27477

§ 371 Date: Oct. 10, 2013

Related U.S. Application Data
(60) Provisional application No. 61/449,283, filed on Mar. 4, 2011.

(51) Int. Cl. G06F 19/16; C12Q 1/68

(52) U.S. Cl. CPC G06F 19/16; C12Q 1/6874
USPC 702/19; 506/2

(57) ABSTRACT

A method of identification of drug targets and drug resistance mechanisms in human cells of a drug comprising the steps of: generating at least one drug-resistant sample and at least one drug-sensitive sample; analyzing substantial portions of the genome and/or transcriptome of the least one drug-resistant sample and drug-sensitive sample to obtain sequencing data; detecting substantially all alterations in the at least drug-resistant sample; deriving a resistance signature; and performing analysis of the drug resistance signature of at least one recurrently altered gene using bioinformatic tools and cellular biology methods to determine if alteration of the at least one gene of the drug resistance signature is sufficient to confer at least partial resistance to cells or tissues against the drug.

### REFERENCE TO RELATED APPLICATIONS

This application claims one or more inventions which were disclosed in Provisional Application No. 61/449,283, filed Mar. 4, 2011, entitled “METHOD FOR RAPID IDENTIFICATION OF DRUG TARGETS AND DRUG MECHANISMS OF ACTION IN HUMAN CELLS”. The benefit under 35 USC §119(e) of the United States provisional application is hereby claimed, and the aforementioned application is hereby incorporated herein by reference.

### ACKNOWLEDGMENT OF GOVERNMENT SUPPORT

This invention was made with Government support under Grant No. 1054964 awarded by the US National Science Foundation CAREER, under Grant Nos. GM98579 and GM65933 awarded by the US National Institutes of Health. The government has certain rights in the invention.

### BACKGROUND OF THE INVENTION

1. Field of the Invention

The invention pertains to the field of identification of drug targets and drug mechanisms. More particularly, the invention pertains to identification of drug targets and drug mechanisms of action in human cells.

2. Description of Related Art

One of the major problems in developing drugs or chemical probes for human treatment is the difficulty in finding their physiological targets. The ‘gold standard’ in identifying a drug's target is achieved in two steps. First, resistance to a drug is identified in a physiological context and can be shown to occur through one or more mutations in the candidate target protein. Second, to establish a direct target, the mutations should suppress inhibition of the target's activity by the drug. This standard is met for a few drugs, however, the large size and complexity of the human genome has limited the unbiased analyses of genetic mechanisms conferring drug resistance in human cells.

Currently, approaches to analyze how a drug works fall into two categories. In a first category, strategies rely on model organisms that are compatible with genetic manipulations. However, many drugs active in human cells are inactive in these models organisms, possibly due to multi-drug resistance mechanisms and target divergence.

In a second category, a variety of affinity-based methods are used to identify proteins that bind the drug. These approaches are generally effective when the drug is potent and the targets are reasonably abundant in vivo. However, proving that a drug-binding protein is the relevant physiological target depends on correlations between chemical inhibition in vitro and protein knockdown phenotypes. These correlations can often prove to be misleading for several reasons, including differences between activity inhibition, which can be acute, and the phenotypes associated with loss of a protein, which can be indirect and/or have cumulative effects.

Analyzing physiologically relevant drug resistance mechanisms remains highly challenging for several reasons. First, the resistance mechanisms can be diverse. These mechanisms can involve mutations in a drug's binding site in its target protein, amplifications of pathways that can bypass the inhibited cellular signals, expression of genes that reduce drug accumulation in cells, and presence of poorly responding cell states (e.g. senescence). Second, as the genomes of certain cells can often be unstable there can be significant heterogeneity in the drug response of these cells, even those that may be present in a single tumor. Third, drug resistance mechanisms can be cell autonomous, or dependent on a cell's microenvironment. Fourth, these mechanisms can be specific to a particular drug (e.g. drug's chemical composition can determine the type of efflux pump required to reduce its accumulation in cells). Fifth, the unanticipated or undesired targets (i.e. off-targets) of drugs vary with chemical structure, even when the drugs share a desired target (i.e. on-target). The contributions of these off-targets to drug resistance (or action) are dose-dependent and can be antagonistic or synergistic, complicating analyses. Determining the mechanisms of drug resistance is critical for developing effective therapeutic strategies that go beyond providing a temporary reprieve from the disease and provide long-term benefits for patients. However, the analysis of the factors contributing to drug resistance is currently almost always biased, ad hoc and retrospective.

The analysis of drug resistance is typically focused on a small set of candidate mechanisms, or the ‘usual suspects’ i.e., the predicted drug target (or related pathways) and multi-drug resistance mechanisms (e.g. drug efflux pumps). However, a drug's physiological target may not be the one predicted and indirect resistance mechanisms can be highly complex. The usual first step involves examining mutations in the drug's direct target. When no mutations can be found in the target, potential indirect mechanisms, such as any known signaling pathways that can effectively bypass the inhibition of the target are examined.

Other indirect mechanisms usually examined include multi-drug resistance, often due to drug-efflux pump over-expression. However, the biased analysis of these candidate mechanisms can often fail in determining the relevant resistance mechanism for many reasons. First, focusing the analysis of resistance on the anticipated target can be risky when the proof of drug activity is based largely on biochemical studies with purified recombinant proteins in vitro (e.g., kinase inhibition in vitro) and correlations with target loss-of-function studies (e.g. RNAi) in cultured cells. This data can be unreliable for reasons that include the now well-documented differences between pharmacological inhibition, which is acute, and loss of protein in cellular contexts or in model systems, which is only achieved on much longer timescales. Second, resistance through indirect mechanisms can involve changes in cellular pathways that can compensate for the loss of signaling due to target inhibition. In most cases, we lack the proper understanding of complex cellular networks involved and often fail to reliably predict and identify the pathways that confer resistance. Third, analysis of multi-drug resistance (MDR) mechanisms focuses on drug-efflux pumps. There are many known drug efflux pumps in human cells that can have redundant functions. Furthermore, the MDR response may involve stress response pathways or changes in cell states. Fourth, many studies rely uniquely on in vitro, non-physiological systems such as cell lines to analyze resistance mechanisms. Together, these factors complicate the proper analysis of these mechanisms and bias on one mechanism is not justified.

Some analysis regarding drug resistance has been carried out in bacteria. For example, Howden et al., entitled, “Genomic Analysis Reveals a Point Mutation in the Two-Component Sensor Gene graS that leads to Intermediate Vancomycin Resistance in Clinical Staphylococcus aureus” discloses a comparative genomic and genetic approach to show that a single base substitution increases vancomycin resistance in an initially vancomycin-susceptible isolate from a patient who initially had an MRSA surgical wound infection but who subsequently developed MRSA endocarditis and persistent bacteremia despite 42 days of vancomycin therapy. Howden et al. uses an already present resistant strain to identify mechanisms of action of vancomycin. Howden et al. then sequences one strain of Staphylococcus aureus to determine the isogenic pair of clinical isolates obtained from a patient before and after vancomycin treatment failure, a mutation that affects a putative sensor histidine kinase, encoded by graS was identified. Allelic replacement in that a single amino acid substitution within the histidine kinase domain was shown to be a major factor in the emergence of the hVISA/VISA phenotype from a vancomycin-susceptible strain.

While Howden et al. did discover the single amino acid substitution within the histidine kinase domain was a major factor in the emergence of the hVISA/VISA phenotype from a vancomycin-susceptible strain, their approach would not have worked on the human genome because they sequenced only 1 strain of Staphylococcus aureus and found 6 mutations in a bacteria whose genome is 2.8 Mb long. In comparison, the human genome is >1,000 times larger. Just multiplying the numbers, sequencing the entire human genome of a resistant clone as done in Howden et al. would give rise to hundreds or thousands of passenger mutations and you could not tell which one is in the target. Furthermore, sequencing coverage in human cells would be lower, and the mutation detection process would need to be significantly more rigorous. Additionally, the resistance being sought is to bacterial drugs and not drugs for humans or mice.

Therefore, there is a need in the art for a method of identification of drug targets and drug resistance mechanism in human cells of a drug.

### SUMMARY OF THE INVENTION

According to one embodiment, a method of identification of drug targets and drug resistance mechanisms in human cells of a drug. The method comprising the steps of: generating at least one drug-resistant sample, comprising at least one drug-resistant cell, wherein the at least one drug-resistant cell in the sample is substantially resistant to the drug and wherein the at least one drug-resistant sample is obtained in vitro from an immortalized normal cell line, a transformed cell line or a disease cell line or the at least one drug-resistant sample is obtained in vivo from immortalized normal tissue or disease tissue; generating at least one drug-sensitive sample, comprising of at least one drug-sensitive cell, wherein the at least one drug-sensitive cell in the sample is sensitive to the drug and wherein the at least one drug-sensitive sample is obtained in vitro from an immortalized normal cell line, a transformed cell line or a disease cell line or the at least one drug-resistant sample is obtained in vivo from immortalized normal tissue or disease tissue; analyzing substantial portions of the genome and/or transcriptome of the least one drug-resistant sample to obtain sequencing data using one of the following methods from the group consisting essentially of: exomic sequencing, genomic sequencing, transcriptome sequencing, epigenomic sequencing, and high-throughput sequencing; analyzing substantial portions of the genome and/or transcriptome of the least one drug-sensitive sample to obtain sequencing data using one of the following methods from the group consisting essentially of: exomic sequencing, genomic sequencing, transcriptome sequencing, epigenomic sequencing, and high-throughput sequencing; detecting substantially all alterations in the at least drug-resistant sample by comparing the sequencing data for the at least one drug-resistant sample to sequencing data of the at least one drug-sensitive sample; deriving a resistance signature by merging the alterations and genes affected by the alterations from the at least one resistant sample and substantially similar resistant cells of the at least one resistant sample with the filtered and identified data generated from the detection of alterations of the at least one resistant sample to obtain a drug resistance signature of at least one recurrently altered gene that has drug resistance across multiple independent resistant cells of the at least one resistant sample; and performing analysis of the drug resistance signature of at least one recurrently altered gene using bioinformatic tools and cellular biology methods to determine if alteration of the at least one gene of the drug resistance signature is sufficient to confer at least partial resistance to cells or tissues against the drug.

According to another embodiment of the present invention, a method of identification of drug targets and drug resistance mechanisms in human cells of a drug using substantial portions of the genome and/or transcriptome of at least one drug-resistant sample to identify substantially all alterations in the at least one resistant sample. The method comprising the steps of: deriving a resistance signature by merging data derived from substantially similar drug-resistant samples with reduced drug sensitivity to the drug and merging the alterations obtained from the substantially similar drug-resistant samples to obtain a drug resistance signature of at least one recurrently altered gene and its alterations that has drug resistance across the drug-resistant samples and sorting the genes and alterations by how frequently the genes and alterations were independently obtained from the substantially similar drug-resistant samples and prioritizing the genes and alterations that are most frequently found; analyzing the drug resistance signature of at least one recurrently altered gene using bioinformatic tools and/or cellular biology methods to determine if alteration of the at least one gene of the drug resistance signature is sufficient to confer at least partial resistance to cells against the drug; and identifying at least one drug target or at least one drug mechanism from the drug resistance signature of a drug that is sufficient to confer at least partial resistance to cells and/or tumors against the drug.

### BRIEF DESCRIPTION OF THE DRAWING

FIG. 1 shows a flowchart of a method of rapid identification of drug targets and drug mechanisms of action in human cells.

FIG. 2 shows a schematic of the steps for a method of rapid identification of drug targets and drug mechanisms of action in human cells of the present invention.

FIG. 3 shows a schematic of derivation of drug-resistant tissues.

FIG. 4 shows a schematic of a pipeline for deriving resistance signatures.

FIG. 5 shows a characterization of BI-2536-resistant clones versus normalized cell growth, where n=3, mean±s.d.

FIG. 6 shows expression levels of ABCB1 mRNA in the parent HCT-116 cells and six BI-2536-resistant clones, with the levels being measured as the number of reads per kilobase, per million reads (RPKM).

FIG. 7 shows a graph based analysis of similarities between BI-2536-resistant clones, with 0 equal to low similarity and 2 equal to high similarity.

FIG. 8 shows a typical monopolar spindle in HCT-116 cells associated with BI-2536 treatment.

FIG. 9 shows bipolar spindles in drug-resistant clone B.

FIG. 10 shows amino acids Arg136 and Gly63 in PLK1, both mutated in BI-2536-resistant clones, adjacent to the BI-2536 binding site in PLK1 (protein databank (pdb): 2RKU).

FIG. 11 shows a proliferation assay showing the effect of 20 nM taxol on HCT-116 parental cells and clones D, E, and F, normalized to untreated cells.

FIG. 12 shows reads mapping at nucleotide 458 of a PLK1 RefSeq transcript in HCT-116.

FIG. 13 shows reads mapping at nucleotide 458 of a PLK1 RefSeq transcript in clone E, R136G.

FIG. 14 shows reads mapping at nucleotide 458 of a PLK1 RefSeq transcript in clone F, R136G.

FIG. 15 shows reads mapping at nucleotide 239 of a PLK1 RefSeq transcript in HCT-116.

FIG. 16 shows reads mapping at nucleotide 239 of a PLK1 RefSeq transcript in clone D, G63S.

FIG. 17 shows the results of a proliferation assay showing the effects of BI-2536 exposure on hTERT-RPE1 cells, stably expressing GFP-PLK1 wild type (WT), GFP-PLK^{G63S} or GFP-PLK^{R136G} with n=6, mean±s.e.m., P<0.01 for the two-tailed paired t-test.

FIG. 18 shows the chemical structure of PLK1 inhibitor BI-2536.

FIG. 19 shows the results of a proliferation assay showing the effects of BI-2536 exposure on HeLa cells transfected with GFP-PLK1 wild type (WT), GFP-PLK^{G63S} with n=4, mean±s.e.m., P<0.05.

FIG. 20 shows the chemical structure of proteasome inhibitor bortezomib.

FIG. 21 shows the lethal dose values measured for the parental cell line and the two drug-resistant clones, clone A and clone E.

FIG. 22 shows a graph based analysis of similarities between bortezomib-resistant clones, with 0 equal to low similarity and 2 equal to high similarity.

FIG. 23 shows the results of a proliferation assay showing the response of hTERT-RPE1 cells, stably expressing GFP-PSMB5 wild type (WT), GFP-PSMB5 M^{104V} or GFP-PSMB5^{A108T} to treatment with bortezomib with n=3, mean±s.e.m., P<0.05 for the two-tailed paired t-test.

FIG. 24 shows the structure of bortezomib with Pre2, a yeast homolog of PSMB5.

FIG. 25 shows the chemical structure of the kinesin-5 inhibitor S-trityl-(1)-cysteine (STLC).

FIG. 26 shows the chemical structure of the kinesin-5 inhibitor 4-(2-(1-phenylcyclopropyl)thiazol-4-yl)pyridine (PCTP).

### DETAILED DESCRIPTION OF THE INVENTION

Drug resistance mechanisms may be linked to alterations in the genomes or the transcriptome of cells. These changes would include point mutations in the drug's target, over-expression of a redundant signaling pathway, or increased expression of drug efflux pumps. These changes could reflect the influence of the cell state or even tissue microenvironment. These alterations would be linked to a particular drug, depending on its chemical composition and mechanism of action, including on- and off-target inhibition.

The term ‘drug target’ is defined as the proteins, cell pathways, and/or mechanisms in which the drug acts.

The term ‘drug resistance’ is defined as any protein, cell pathway or mechanism which reduces drug efficacy.

The term ‘drug resistance cell’ is defined as a cell that is substantially more resistant than control or parental cells and may reduce drug efficacy. The cells can be tumors.

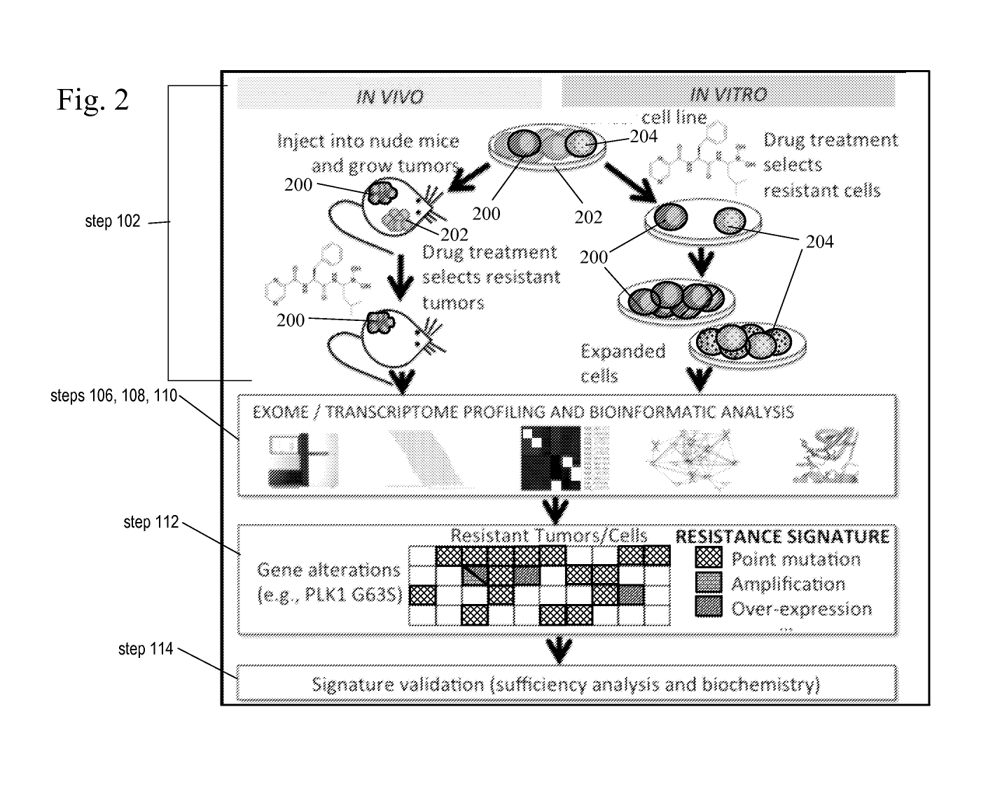

FIG. 2 shows a schematic overview of a portion of the method of the present invention of rapid identification of drug targets and drug resistance mechanisms in human cells.

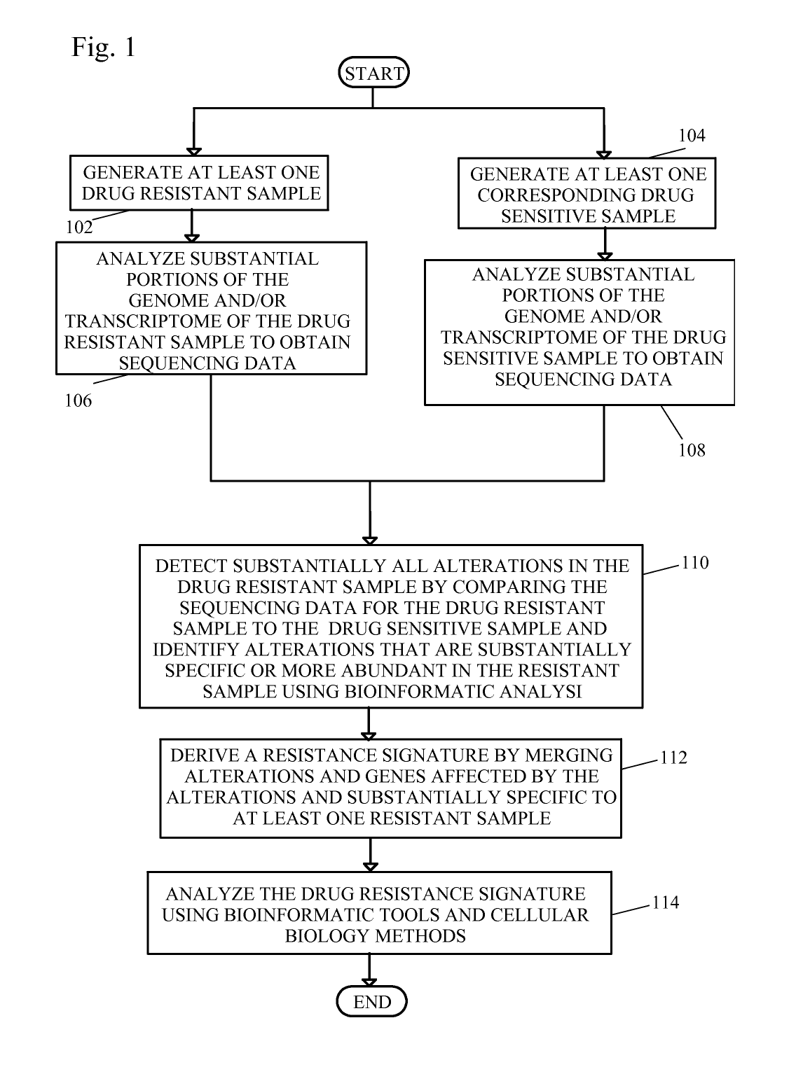

A drug-resistant sample is generated (step 102—FIG. 1) by obtaining at least one drug-resistant cell using drug-resistant clones 204, 200 isolated from cell lines 202 in culture in vitro and/or from in vivo tissues, for example isolated drug-resistant mouse tumor xenografts 200, treated with a drug. The cell line used to generate the resistant clones and the resistant tumors may be the same type cell line. The cell lines 202 used to obtain at least one drug-resistant cell in vitro may be a transformed cell line, immortalized normal cells (referred to as wild type), or a disease cell line. The cell line 202 used to obtain at least one drug-resistant cell in vivo may be immortalized tissue or disease tissue. The tumors used may be primary tumors, in which the tumor is at the original site where it first arose or a secondary or metastatic tumor, in which the tumor has moved from the original site to another non-adjacent organ or part.

If immortalized normal cells are used, a mutagenesis agent, such as N-ethyl-N-nitrosourea (ENU) may be used in conjunction to induce mutations. Sub-maximal (or sub-optimal) drug dose may be administered to obtain resistant cells and/or tumors.

Furthermore, the drug-resistant sample be derived by growing cells in vitro at doses close to, but lower than a measured lethal dose of the drug. The drug-resistant sample may be derived by selecting cells that express a marker, reporter gene or phenotype that indicates that a cell is resistant or sensitive to the drug.

The drug-resistant sample may also be obtained by injecting at least one disease cell from a cell line into at least one animal, the at least one disease cell may divide within the at least one animal, the at least one animal is treated with the drug continuously or using multiple on/off treatment cycles so as to select for samples with reduced sensitivity to the drug, and the at least one resistant sample is collected from the at least one animal. Alternatively, the drug-resistant sample may be derived by treating at least one animal with the drug, where the at least one animal may be genetically engineered or not and collecting the at least one resistant sample from the at least one animal.

In another embodiment, the resistant sample is derived from at least one human treated with the drug and at least one resistant cell is collected from the human.

The drug-sensitive sample may be obtained from cells with substantially similar genetic background as the drug-resistant sample, and collected in vitro or in vivo.

Since some resistance mechanisms will be found in cells grown in both culture and in tumors, while others will be unique to the cells grown in culture or in the tumors, preferably priority is given to resistance mechanisms observed in tumors. The combined in vitro and in vivo analysis is beneficial, since it provides information on which resistance mechanisms can be modeled and further investigated using cell lines but not necessary for the method. Moreover the in vitro data provides information regarding all the direct and indirect targets of a drug, including data on selective pressure at play within tumors that may prevent or favor the emergence of certain resistance mechanisms.

At least one corresponding drug-sensitive sample is also generated from the cell line used (step 104—FIG. 1). The drug-sensitive sample has at least one drug-sensitive cell which can be generated from in vivo or in vitro analysis. The cell lines used to obtain at least one drug-sensitive cell in vitro may be a transformed cell line, immortalized normal cells (referred to as wild type), or a disease cell line. The cell line used to obtain at least one drug-sensitive cell in vivo may be immortalized tissue or disease tissue.

Next, an analysis pipeline may be used to identify genomic alterations (e.g. point mutations, indels, copy number variations, gene fusions and gene expression differences) present in the drug-resistant sample, but not detected in the drug-sensitive sample through exome, transcriptome profiling and bioinformatic analysis (steps 106; 108; 110 see FIG. 1). The output from the analysis will be a drug ‘resistance signature’ (steps 112, 114; see FIG. 1), that is, a set of gene alterations recurrently found across multiple independent resistant cells in one drug-resistant sample or multiple drug-resistant samples.

Each alteration in a resistance signature may be validated by expressing it in the drug-sensitive cell of the drug-sensitive sample and determining whether it can confer even partial resistance (step 116; see FIG. 1). Further biochemical and cell biology approaches may be used to determine if the drug resistance mechanisms, detected at high frequencies in independent isolates, are directly linked to mutations or amplifications of a drug target, or are indirect and involve pathways that can overcome target inhibition.

The drug resistance signature may also be used to aid in patient treatment. For example, the drug resistance signature may be used to anticipate drug resistance in patients by predicting drug resistance mechanisms and chemically modifying a drug or altering drug usage parameters to reduce expected resistance. The drug resistance signature may also be matched to a patient's genomic data to design a therapeutic strategy that is appropriate in light of the mutations the patient may have, for example drug usage. The drug resistance signature can also be compared to genomic data for a human patient to provide patient prognostics related to drug efficacy. The drug-resistant signature may be used to provide prognostics by knowing what existing mutations are present in patients that would indicate if a drug will work or not. The drug-resistant signature may also be used to determined the drug toxicity in healthy tissue of patient, since the drug targets that lead to toxicity will be revealed within the drug resistance signature and chemistry can be used to modify drugs to address this. The drug resistance signature may also be used to analyze interactions of a drug with a potential target by generating analogs for immobilization for chemical synthesis.

Referring to FIGS. 1-2, more specifically, generate at least one drug-resistant sample (step 102). The drug-resistant sample includes at least one drug-resistant cell which is obtained in vivo or in vitro from a cell line. The cell line used to obtain at least one resistant cell in vivo may be from immortalized normal tissue or disease tissue. The cell line used to obtain at least one resistant cell in vitro may be from an immortalized normal cell line, a transformed cell line or a disease cell line. It should be noted that the method of the present invention may be used with cell lines from humans, mammalians or fish.

For example, the cell lines may be cancer cell lines, for example, BRCA1/2 deficient cell lines, such as HCC1937 and EUFA423 to study resistance. Other cell lines may also be used depending on what resistance is being targeted and what is amplified within the cell line. For example, cell lines such as MDA-361, UACC-812 may be used to study resistance to HER-2 targeted inhibitors, since HER-2 is amplified in these cells. If resistant cells cannot easily be obtained from the cells lines, lentiviruses may be used to generate stable lines with shRNA-mediated knock down of mismatch repair genes.

It should be noted that in vitro resistant cells may be derived by growing cells at doses close to, but lower than, the measured lethal dose LD₅₀s or a non-lethal dose. Clones can then be expanded in the presence of a drug. At least 10 or more clones may be chosen per drug, thus significantly increasing the number of clones and the potential diversity of resistance mechanisms.

In addition to transcriptome sequencing, exome sequencing may be performed on these resistant cells, increasing the ability to uncover resistance-conferring mutations in non-expressed genes or genes expressed at low levels, including potential nonsense mutations that may give rise to non-sense mediated RNA decay.

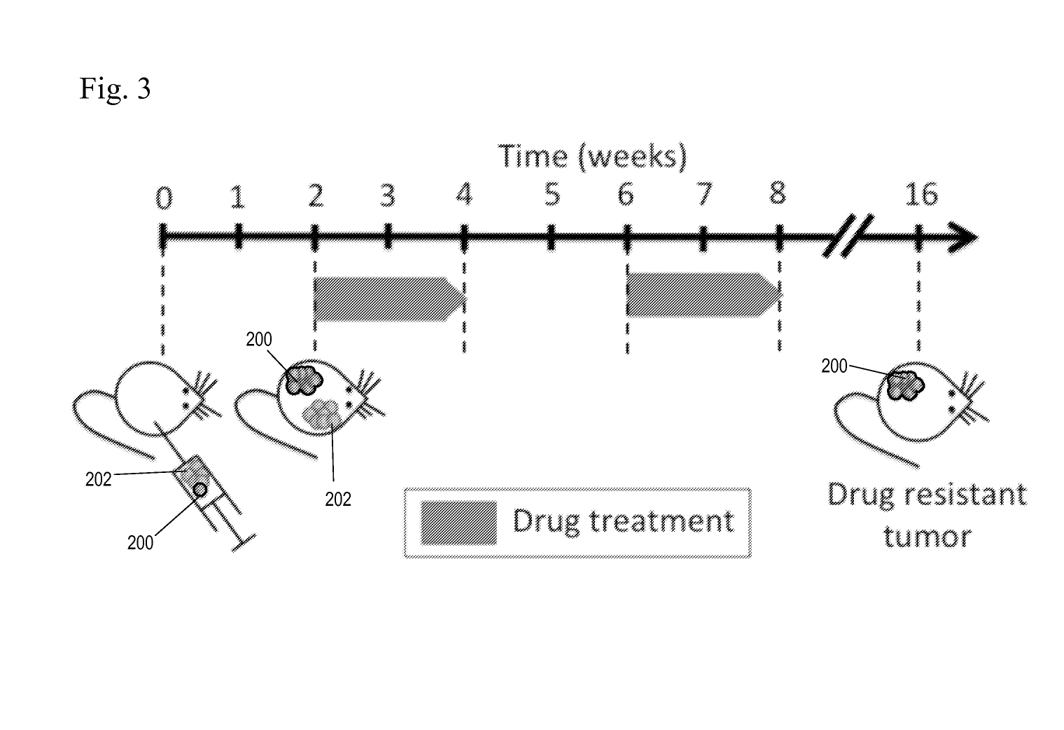

Resistant cells generated in vivo may be from tumors. For example, mice may be injected with the appropriate cell line 200, 202, 204 and later injected with a drug of choice to obtain drug-resistant cells, for example tumors. As shown in FIG. 3, cells 200. 202 from a cell line are injected into mice at week 0. The mice with papable tumors, obtained by week 2, are randomized into two groups, a treatment group and a control group. The treatment group preferably receives the drug of choice for two weeks (weeks 2-4), taken off treatment for two weeks (weeks 4-6) and then treated again for two weeks (weeks 6-8), etc. . . . for a number of cycles. The mice in the control group are treated with a control for the same frequency. The size of the tumors of the mice may be quantified through size and volume and a qualification standard may be implemented. The tumors from both the control group and the treatment group may then be surgically removed.

Then, sequencing data from the drug-resistant cells which may include the drug-resistant cells from the in vitro analysis 102 and the drug-resistant cells or tumors from the in vivo analysis is obtained for genomic analysis (step 106).

At least one corresponding drug-sensitive sample is also generated (step 104). The drug-sensitive sample includes at least one drug-sensitive cell which is obtained in vivo or in vitro from a cell line. The cell line used to obtain at least one sensitive cell in vivo may be from immortalized normal tissue or disease tissue. The cell line used to obtain at least one drug-sensitive cell in vitro may be from an immortalized normal cell line, a transformed cell line or a disease cell line. While this step is shown as being after step 102, this step may take place before step 102 or simultaneously with step 102.

Then, sequencing data from the drug-sensitive cells which may include the drug-sensitive cells from the in vitro analysis 102 and the drug-sensitive cells or tumors from the in vivo analysis is obtained for genomic analysis (step 108).

For example, the drug-resistant cells of the drug-resistant sample and the drug-sensitive cells of the drug-sensitive sample may be processed for genomic and transcriptomic single-base resolution profiling. The cells may be dissociated, and DNA and RNA extracted using standard procedures, such as DNA and RNA extraction kits, for example DNeasy and RNeasy Qiagen kits. The quality of the DNA and RNA may be tested, for example using the Bioanalyzer from Agilent Technologies. After the quality of the DNA and RNA has been confirmed, exome capture may be performed, for example using Agilent Technologies' SureSelect platform; and a cDNA library construction may be performed using procedures such as described in methods below. Following library preparation, sequencing may be performed, for example using an Illumina HiSeq2000 platform.

The tools and test discussed and performed are just examples of what types of tools may be in genomic analysis of the samples. Other tests that provide information regarding the profile of the samples may also be used without departing from the scope of the invention.

Next, substantially all alterations from sequence data in the drug-resistant sample are detected by comparing the sequence data of drug-resistant sample to the drug-sensitive sample and identifying alterations that are substantially specific or more abundant in the drug-resistant sample using bioinformatic analysis (step 108).

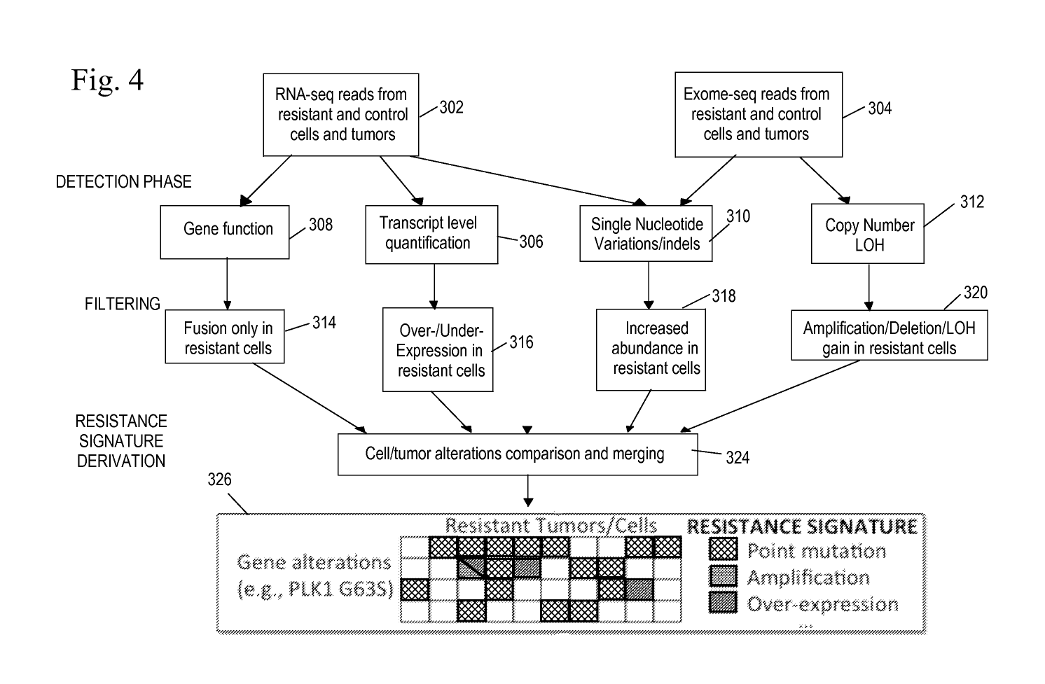

For example, as shown in FIG. 4, RNA-seq reads 302 and exome-seq reads 304 may be used to perform the following analyses in resistant tumors and cells as well as well as in control samples in the detection phase to: estimate transcript levels 306, identify gene fusions 308 identify candidate variations 310, i.e., point mutations and short indels, and copy number quantification and loss of heterozygosity (LOH) detection 312. Genes may be differentially expressed by fusion only in resistant cells 314, over-/under-expression in resistance cells 316, increased abundance in resistant cells 318, and through amplification/deletion, LOH gain in resistant cells 320. Tools and tests may be used to filter and identify what genes are differentially expressed.

For transcript level estimation 306, TopHat, a fast splice junction mapper for RNA-seq reads, for example, may be used to map reads to the human genome (hg19). CuffLinks, a program that assembles transcripts, estimates their abudnaces and tests for differential expression and regulation in RNA-seq samples may then be used to estimate gene expression levels (FPKM) using upper-quartile and guanine-cytosine level (GC-level) normalization.

To determine over-/under-expression in resistant cells 316, DEseq, a computer program with a Benjamini-Hochberg correction for multiple testing may be used to analyze count data from high-throughput sequencing assays such as RNA-Seq and test for differential expression.

Identification of single nucleotide variants (SNVs) and indels 310 may be performed using SNVseeqer, a program for SNV discovery and characterization from RNA-seq and DNA-seq data and INDELseeqer, a program for indels discovery. Other detection methods and tools such as SNVmix, a tool which detects single nucleotide variants from next generation sequencing data; VarScan, a platform-independent, technology-independent software tool for identifying SNPs and indels in massively parallel sequencing of individual and pooled samples; GATK, structured software library that makes writing efficient analysis tools using next-generation sequencing data very easy, and second it's a suite of tools for working with human medical resequencing projects such as 1000 Genomes and The Cancer Genome Atlas; and DINDEL, takes BAM files with mapped Illumina read data and enables researchers to detect small indels and produce a VCF file of all the variant calls; may also be used and preferably only variants detected by more than one approach are used for a determinative list of SNVs and indels. Sanger sequencing may then be used to validate SNV and indel detection.

To determine increased abundance in resistant cells 318, for example whether SNVs and indels 318 are more abundant in resistant cells 200, 204 compared to sensitive tumors cells, Fisher exact tests may be performed, preferably with a controlled FDR=5%. This type of analysis allows detection of variants that may be already present in the control cells/tumors but at lower abundance than in the resistant cells. SNVs and indels may also be detected even if the tumors contain multiple populations of resistant and sensitive cells.

Gene function 308 impacts i.e., missense, nonsense, frameshift leading to premature stop codon may be detected using Blocks of Amino Acid Substitution Matrix (BLOSUM) to obtain a BLOSUM score, for example a BLOSUM62 score. Other tools that may be used to determine gene function impact are PolyPhen, a tool which predicts a possible impact of an animo acid substiction on the structure and function of a human protein and sorting intolerant from tolerant (SIFT), a program that predicts whether an amino acid substitution affects protein function. In addition, the RNA-seq reads may be screened against databases. For example against the Catalogue of Somatic Mutations in Cancer (COSMIC) to reveal if these resistance conferring mutations are known to occur in cancer patients. Altogether, preferably in testing, only variations either previously known in COSMIC or predicted to have a reasonably deleterious effect on gene function (e.g., with at least BLOSUM62 score≧0) should also be retained.

To determine gene fusions in resistance cells 314, absent in sensitive cells, TopHat-Fusion which is a program with the ability to align reads across fusion points, which results from the breakage and re-joining of two different chromosomes, or from rearrangements within a chromosome may be used, along with FusionSeq, a computational framework which identifies fusion transcripts from paired-end RNA-sequencing. If high-confidence fusions are found in resistant cells, i.e., fusions supported by multiple reads mapping across the fusion junction, the fusions may be validated using polymerase chain reaction (PCR) with primers designed to amplify the junction.

To detect copy number variations (CNVs) 320, DEseq, an R package that analyses count data from high-throughput sequencing assays such as RNA-Seq and test for differential expression, may be used to detect amplification and deletion at the gene and exon level. Preferably, circular binary segmentation on exon-level read count log ratios may be used to detect large-scale genome rearrangement.

Potential loss of heterozygosity (LOH) 320 may be detected by comparing the fraction of homozygous SNPs and SNVs within each gene to the same quantity obtained from the sequencing of genomes of a large number of people, for example as provided by the 1000 Genome project. The result of the comparison may then be assessed using Fisher Exact tests, a statistical significance test used in the analysis of contingency tables. Gene copy numbers may be estimated from Burrows-Wheeler Aligner (BWA), a program that aligns relatively short nucleotide sequences against a long reference sequence such as the human genome, to align read counts and preferably circular binary segmentation may be used to find segments of equal copy number.

The tools and test discussed and performed are just examples of what types of tools may be used to detect substantially all alterations in the drug-resistant sample in comparison to the drug-sensitive sample and identify alterations that are substantially specific or more abundant in the resistant sample using bioinformatic analysis. Other tests that provide information regarding detecting substantially all alterations in the drug-resistant sample in comparison to the drug-sensitive sample and identifying alterations that are substantially specific or more abundant in the resistant sample using bioinformatic analysis may also be used without departing from the scope of the invention.

Then, a resistance signature is derived by merging alterations and genes affected by the alterations and substantially specific to at least one resistant sample (step 112). The definition of the term ‘substantially similar’ or ‘substantially specific’ means exhibiting similar resistance to the same drug. The term ‘substantially similar’ or ‘substantially specific’ does not take into account as to whether the actual resistant cells are the same.

In one embodiment, the alterations and genes affected by the alterations from the resistant sample are merged with other and substantially similar resistant cells of the same resistant sample to derive a drug resistance signature of at least one recurrently altered gene that has drug resistance across multiple independent resistant cells of the sane resistant sample.

In another embodiment, the alterations and genes affected by the alterations from a first drug-resistant sample and a substantially similar second drug-resistant sample with filtered and identified data generated from the detection of alterations are merged to obtain a drug resistance signature of at least one recurrently altered gene that has drug resistance across multiple independent drug-resistant samples.

Since resistant cells are not always genetically independent (even if picked from different plates), the non-independent clones can give rise to a false positive recurrent mutation. A hypergeometric graph-based approach may be applied to identify identical cells and tumors and merge the results together 324 as a drug ‘resistance signature’ 326. Therefore, the drug ‘resistance signature’ 326 are the genes and alterations that have been uncovered more than once in independent resistant cells or samples. Therefore, the drug ‘resistance signature’ potentially includes or leads to the relevant ‘drivers’ of drug resistance, excluding the ‘passenger’ genomic and transcriptional alterations.

The tools and test discussed and performed are just examples of what types of tools may be used to derive a drug resistance signature. Other tests that provide information ways of deriving a resistance signature may also be used without departing from the scope of the invention.

Next, the drug resistance signatures of at least one recurrently altered genes are analyzed through bioinformatic tools and cellular biology methods to determine if the alteration of at least one gene of the drug resistance signature are sufficient to confer at least partial resistance against the drug (step 114).

Some examples of bioinformatic tools that may be used for network analysis and functional analysis of the drug resistance signature are STRING, a database of known and predicted protein interactions including direct/physical and indirect/functional associations, DAVID, a database of functional annotation tools for investigators to understand biological meaning behind large list of genes, and iPAGE, an integrated platform for exploring large-scale gene expression and protein behavior dynamics.

Part of the network and functional analysis may also include mapping point mutations and indels onto any available 3D protein structure to examine how they may interfere with the biochemical activity of these proteins. These analyses can aid in determining whether specific pathways or networks are frequently associated with resistance and generate hypotheses for what their alterations may do. These pathway-level analyses are of value since individual genes may be mutated at low frequency across tumors/cells but may belong to the same pathway, indicating that the pathway itself is responsible for resistance in physiological contexts

To determine if gene alterations of the ‘resistance signature’ are sufficient to confer resistance, one of the following two strategies may be used: (1) for genes in which mutations are identified, stably express this allele in the cancer cell line using retroviral based systems; and (2) for a gene that is over-expressed, use RNAi-mediated knockdown in isolated (and expanded) drug-resistant clones. Then, the dose-dependent drug sensitivity in culture may be examined. It is possible that some drug resistance conferring changes that are identified only in the mouse studies and may not be readily recapitulated in cell lines grown in culture. For these analyses, it may be necessary to either inject the cell lines stably expressing the mutant allele, or cell lines carrying an inducible shRNA targeting the over-expressed gene.

It is possible that an observed mutation is present in the drug's anticipated target. If expression of this mutant allele and is sufficient to confer drug resistance, biochemical assays, such as in vitro activity (e.g. substrate phosphorylation) and/or binding (e.g. isothermal calorimetry or fluorescence anisotropy) may be used to examine if the mutation can alter drug-target interaction. Recombinant wild-type and mutant proteins that may be used will preferably be expressed in bacteria or insect cells. Alternatively, biochemical tests may be carried out using tagged protein complexes isolated from cells stably expressing the wild-type or the mutant protein.

To analyze interactions of a drug with a potential target, chemical synthesis will be used to generate analogs for immobilization (on affinity-matrices), tagging (e.g. fluorescent) or capture (e.g. using CLICK chemistry).

When mutations are observed in a gene that is not the anticipated target, and this mutant allele is found to be sufficient to confer drug resistance, we may examine if this protein is an unanticipated target that directly binds the drug. Wild-type and mutant forms of the gene product, expressed as a recombinant protein in bacteria or insect cells, may be examined using biochemical assays. If the drug is found to bind or inhibit the protein, and the mutation can suppress drug activity in vitro, and the cumulative data already collected may then establish this protein as a physiologically relevant target of the drug.

If no direct interaction of the drug with the protein is observed, it may suggest that the resistance mechanism involves a pathway that can compensate for the loss of target activity. Detailed cell biological analyses, guided by all available data for the target protein and the gene, may then be used to unravel how it confers indirect drug resistance.

Below are examples in which an unbiased ‘resistance signature’ for two chemically and functionally unrelated anticancer drugs were derived: BI-2536, a Polo-like kinase 1 (PLK1) inhibitor in clinical trial for relapsed acute myeloid leukemia; and bortezomib, an FDA-approved proteasome inhibitor used against multiple myeloma and mantle cell lymphoma. In the examples below HCT-116 colon cancer cells grown in culture were used. This cancer cell line is mismatch repair deficient and is therefore genetically unstable, thereby representing a test case that may match the level of genetic heterogeneity that could be observed to analyze network function.

The tools and test discussed and performed are just examples of what types of tools may be used to determine if the alteration of the genes of the drug resistance signature are sufficient to confer at least partial resistance against the drug through network and functional analysis using bioinformatic tools. Other tests that provide information to determine if the alteration of the genes of the drug resistance signature are sufficient to confer at least partial resistance against the drug through network and functional analysis using bioinformatic tools may also be used without departing from the scope of the invention.

It should be noted that Example 2 includes additional work and tests which confirmed the results of Example 1. Additional data was run and the number or samples increased. Furthermore, the error scale in Example 2 was mostly calculated using standard error of the mean (S.E.M.), while the error scale used in Example 1 was standard deviation (S.D.). No significant differences were seen between the data obtained in Example 1 versus Example 2.

### Example 1

The mechanisms that confer resistance to a drug in clinical trials whose target is known was analyzed. The compound that was used was BI-2536, a dihydropteridinone that inhibits Polo-like kinase 1 (PLK1), a major cell cycle regulator. To select drug-resistant clones, the human colon cancer cell line, HCT-116, was used, which is mismatch repair deficient, genetically heterogenous, and does not require many passages in order to find resistance conferring mutations. The HCT-116 cells (500,000 cells per plate, 9 plates total) were treated with 10 nM BI-2536, a concentration at which this drug kills most cells (LD₅₀: 3.9 nM).

Fifteen clones capable of growing in 10 nM BI-2536 were isolated and expanded. Then six clones were randomly selected whose LD₅₀'s were 3-9 fold higher than for the parental cell line and as shown in Table 1. RNA was isolated from each of the six clones, along with the untreated ‘parental’ cells, and processed in parallel for total transcriptome sequencing (referred to as RNA-seq).

TABLE 1
(mean + s.d.)

| Clone | LD₅₀ (nM) |
|---|---|
| Parental | 3.9 ± 1.0 |
| A | 24.7 ± 5.9 |
| B | 22.9 ± 5.3 |
| C | 12.6 ± 2.6 |
| D | 27.6 ± 2.4 |
| E | 35.9 ± 9.9 |
| F | 33.4 ± 7.5 |

The sequencing data, which consisted of 25-29 million 40 bp-long mappable reads per clone, was analyzed as described below in ‘Methods’.

Briefly, single nucleotide variations and short insertions/deletions (indels) were separately identified from each clone. The subset of these variants whose relative abundance was significantly increased (with a 5% false discovery rate) in the clone compared to the parental HCT-116 population was then determined.

Only single nucleotide variations resulting in missense mutations and indels in coding sequences were further considered. This analysis revealed 9-24 single nucleotide variations (but no indels) in each clone. We focused our analysis on genes that were mutated in more than one of the six clones as shown in Table 2 below.

TABLE 2

| | Amino | | | | | | |
|---|---|---|---|---|---|---|---|
| | Acid | Resistant Clone | | | | | |
| Gene | Mutation | A | B | C | D | E | F |
| PLK1 | R136G | | | | | X | X |
| PLK1 | G63S | | | | X | | |
| ARF3 | A27T | X | X | X | | | |
| MACROD1 | P142L | X | X | X | | | |
| PPP1CA | D168G | X | X | X | | | |
| CD151 | Y62C | X | X | | | | |
| PRICKLE3 | R530H | X | | X | | | |
| PYCR1 | A8D | | X | X | | | |
| TCF3 | G302D | | X | X | | | |
| UTP3 | E321G | X | X | | | | |

Based on Table 2, it is shown that decreased sensitivity to BI-2536 can be conferred by two types of mechanisms: mutations in the PLK1 gene (clones D, E, and F) and mutations unrelated to PLK1 (clones A, B, and C).

Analysis of RNA-seq data from the clones that did not have mutations in the PLK1 gene (clones A, B, and C) identified eight genes that were mutated in more than one clone. Identical mutations in three genes, ARF3, MACROD1, and PPP1CA, were present in each of these clones. This data indicated that these clones were derived from a common ancestor. We are able to confirm that clones A, B, and C not only suppress BI-2536-induced cell death, but also the characteristic cell division phenotype associated with loss of Plk1 activity. In particular, the reduction in bipolar spindles, associated with BI-2536 treatment, is suppressed in clones A, B, and C. This raised the possibility that increased drug efflux is a potential resistance mechanism in these clones. An advantage of using massively parallel sequencing is that it is possible to examine transcript levels of all RNAs sequenced. An unbiased survey of all transcripts that were increased or decreased two-fold from the parental HCT-116 population revealed highly elevated levels of ABCB1 (P-gp, a drug efflux transporter) mRNA in clones A, B, and C as compared to the low levels in the parental cells. This is consistent with the effective BI-2536 concentration being actively reduced in these clones. Since clones A, B, and C likely share a common ancestor, it is improbable that the up-regulation of the ABCB 1 transporter occurred independently in these lines. Importantly, clones A, B, and C are less sensitive than the parental cell line to taxol, a compound known to be transported by ABCB 1. This data indicates that our method can reveal indirect drug resistance mechanisms, such as increased drug efflux, which is commonly seen in clinical resistance.

Three other clones, clones D, E, F, had mutations in PLK1 and in these clones ABCB1 levels were not increased. Analysis indicated there were no other mutations common within these clones, or with the other analyzed clones as shown in Table 2 above, which is consistent with the BI-2536-resistance conferring mutations in PLK1 arising independently. Of the genes identified by our analysis, PLK1 was the only one in which two distinct mutations were found (G63S and R136G). These mutations were not detected in the original HCT-116 cell population, despite high expression of PLK1 in these cells. Furthermore, analysis of PLK1 mutations by RT-PCR and Sanger sequencing of the nine clones not subjected to RNA-seq indicates that mutations in the target is a common mechanism of BI-2536 resistance.

The two Plk1 mutations (G63S and R136G) map to the binding site of BI-2536 in the crystal structure of PLK1. G63S, a mutation that has not previously been reported, likely occludes the binding of BI-2536 by replacing a small amino acid with one that has a bulkier side-chain. Interestingly, R136G has been shown to suppress BI-2536 inhibition (IC₅₀ increase of ˜3 fold, in vitro kinase assays), most likely due to loss of favorable interactions of the drug with the arginine side-chain. These mutations must not dramatically alter kinase activity, as their presence rescues cell growth when the wildtype copy of PLK1 is inhibited by BI-2536. We next examined whether these mutations in PLK1 are sufficient for conferring resistance to BI-2536. This test is crucial as mutations in other genes (observed only once) were present in each clone. We expressed these mutations (PLK1 G63S and PLK1 R136G) in the full-length protein as a GFP-tagged construct in an independent cell line (hTERT-RPE1), which was known to be sensitive to BI-2536. It was found that expression of each mutant construct suppressed BI-2536 toxicity. There is evidence that BI-2536 is active against two other members of the Polo-like kinase family (PLK2,3) in vitro. Of these three potential targets, the data indicate PLK1 is the most likely physiologically relevant target of BI-2536.

### Example 2

The mechanisms that confer resistance to a drug in clinical trials whose target is known was analyzed. The specifics of the exact methodology used is discussed below under the title “Methods”. The compound that was used was BI-2536, a dihydropteridinone that inhibits Polo-like kinase 1 (PLK1), a major cell cycle regulator. To select drug-resistant clones, the human colon cancer cell line, HCT-116, was used, which is mismatch repair deficient, genetically heterogenous, and does not require many passages in order to find resistance conferring mutations. The HCT-116 cells (500,000 cells per plate, 9 plates total) were treated with 10 nM BI-2536, a concentration at which this drug kills most cells (LD₅₀: 3.9 nM).

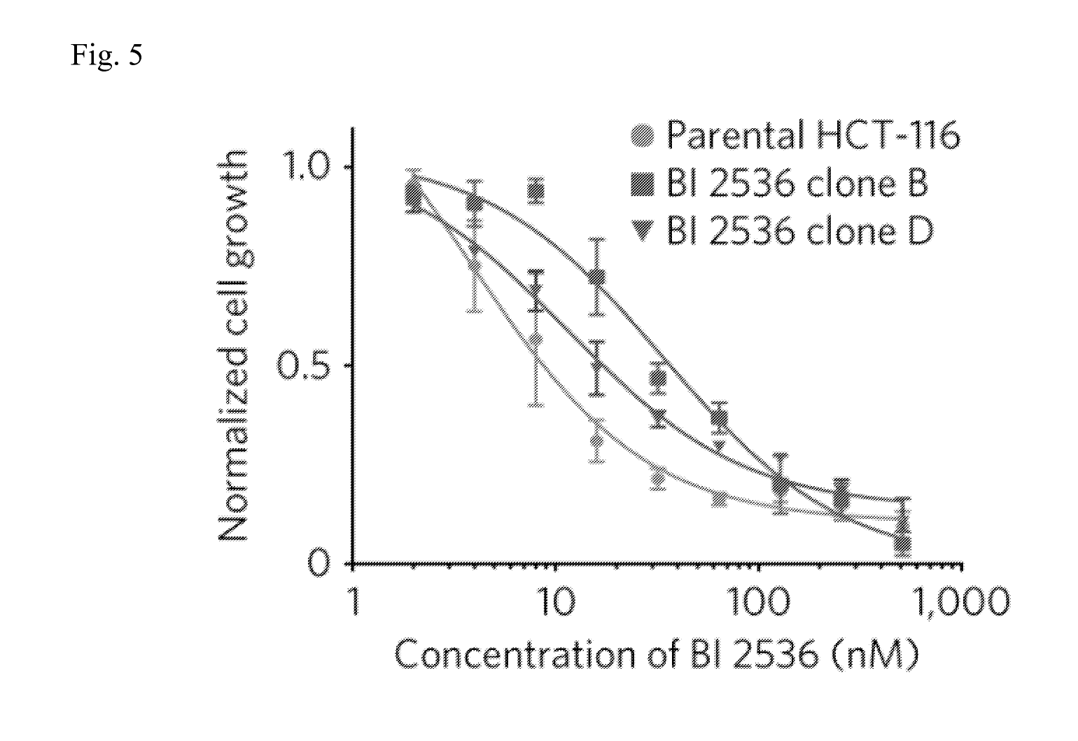

Fifteen clones capable of growing in 10 nM BI-2536 were isolated and expanded. Then six clones were randomly selected whose LD₅₀'s were 3-9 fold higher than for the parental cell line and as shown in Table 3 and FIG. 5 of concentration of BI-2536 (nM) versus normalized cell growth. RNA was isolated from each of the six clones, along with the untreated ‘parental’ cells, and processed in parallel for total transcriptome sequencing (referred to as RNA-seq).

TABLE 3

| Clone | LD₅₀ (nM) |
|---|---|
| Parental | 3.9 ± 2.8 |
| A | 34.9 ± 13.0 |
| B | 33.5 ± 2.6 |
| C | 27.2 ± 8.6 |
| D | 14.2 ± 7.8 |
| E | 21.1 ± 5.7 |
| F | 27.5 ± 13.3 |

where n = 3, mean ± s.d

The sequencing data, which consisted of 25-29 million 40 bp-long mappable reads per clone, was analyzed as described in the methods below.

Briefly, single nucleotide variations (SNV) and short insertions/deletions (indels) were separately identified from each clone. The subset of these variants whose relative abundance was significantly increased (with a 5% false discovery rate) in the clone compared to the parental HCT-116 population was then determined.

Only single nucleotide variations (SNV) resulting in missense mutations and indels in coding sequences were further considered. This analysis revealed 6-14 single nucleotide variations significantly increased (with a 0.5% false discover rate) in BI-2536-resistant clones compared to the parental cell population, but no indels.

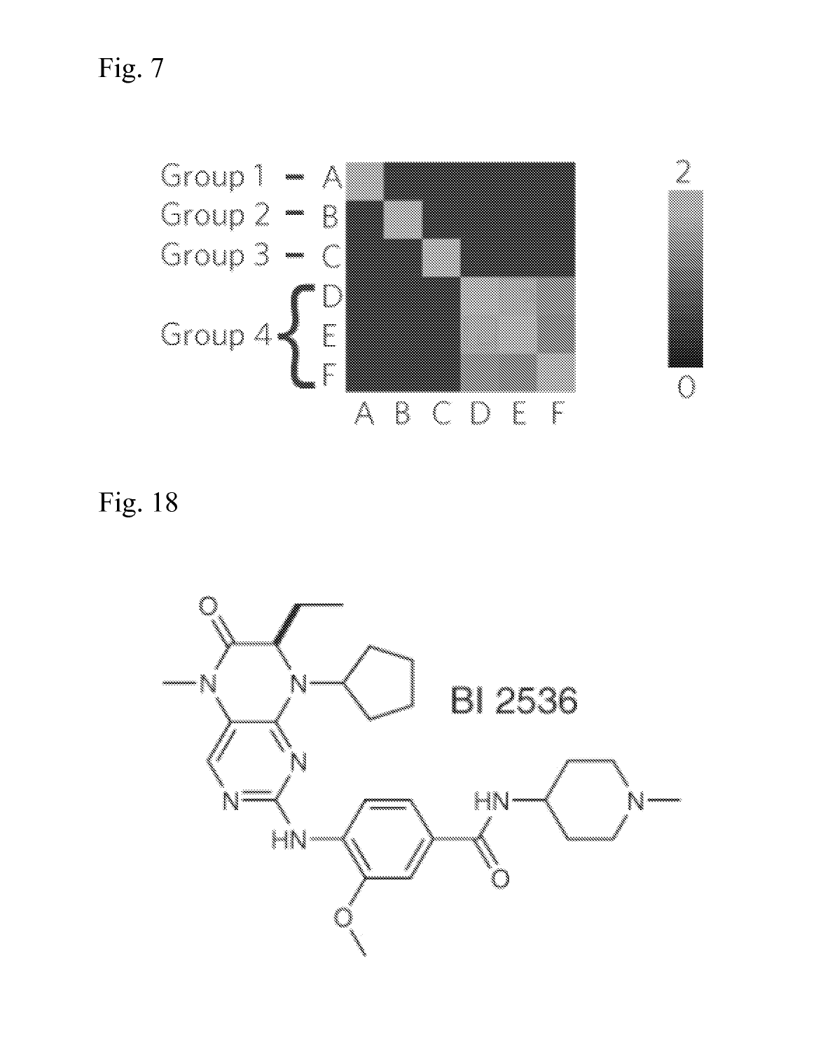

Groups of similar clones were identified by analyzing single nucleotide variation (SNV) using a clustering approach. Among the six clones, clones A, B, and C were independent (groups 1, 2, and 3 respectively), where as clones D, E, and F formed a single group (group 4), as shown in FIG. 7.

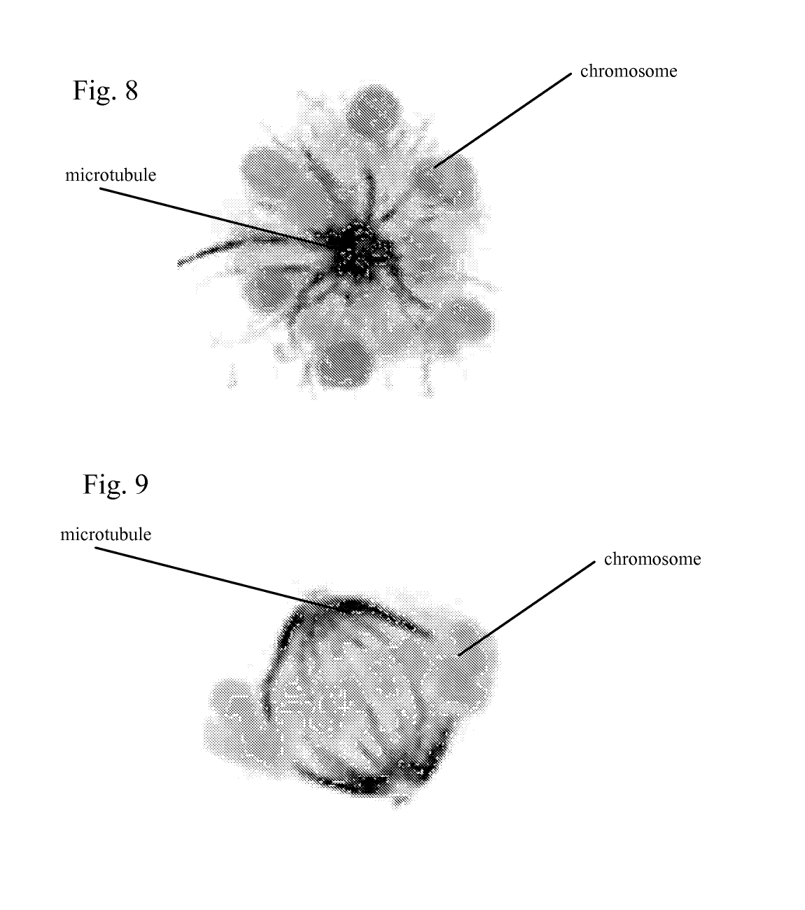

Analysis of RNA-seq data from the clones that did not have mutations in the PLK1 gene (clones D, E, and F) identified eight genes that were mutated in more than one clone. Identical mutations in three genes, ARF3, MACROD1, and PPP1CA, were present in each of these clones. This data indicated that these clones were derived from a common ancestor. We are able to confirm that clones D, E, and F not only suppress BI-2536-induced cell death, but also the characteristic cell division phenotype associated with loss of PLK1 activity, in particular, the reduction in bipolar spindles, associated with BI-2536 treatment. Normal bipolar spindles were observed in only 11±3% of HCT-116 cells. A typical monopolar spindle in these cells is shown in FIG. 8. FIG. 9 shows that in comparison 46±4% of the spindles in the drug-resistant clone B were bipolar. It should be noted that the black background was removed for readability purposes.

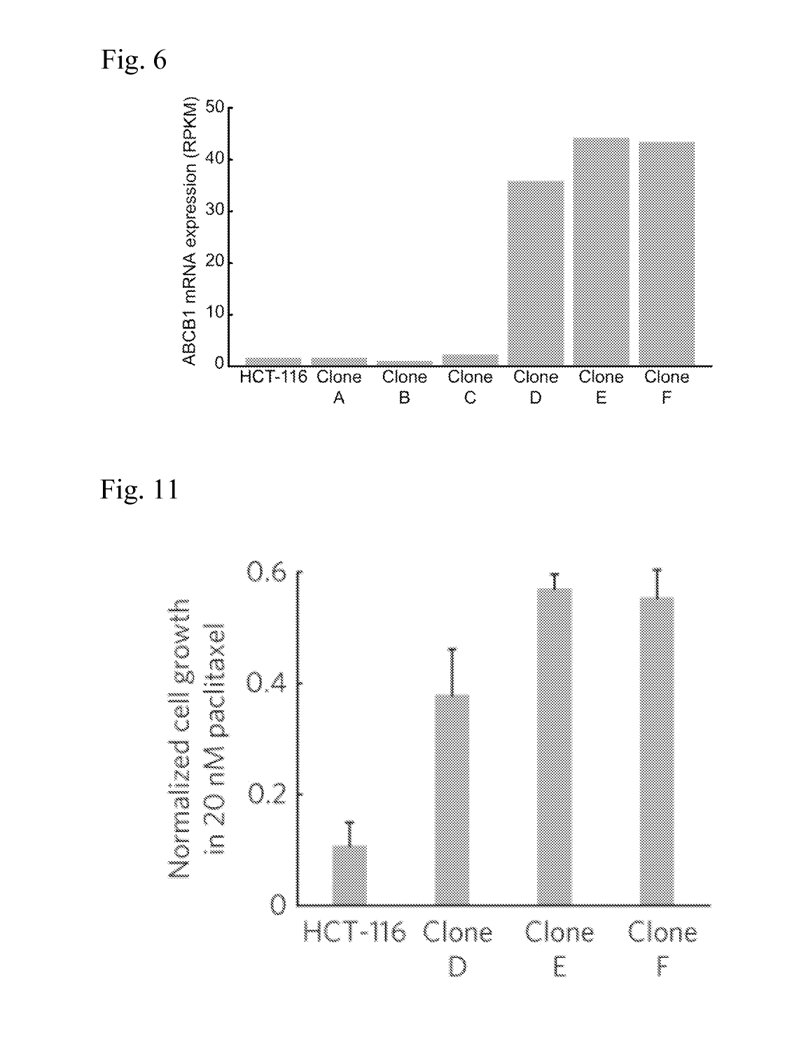

This raised the possibility that increased drug efflux is a potential resistance mechanism in these clones. An advantage of using massively parallel sequencing is that it is possible to examine transcript levels of all RNAs sequenced. An unbiased survey of all transcripts that were increased or decreased two-fold from the parental HCT-116 population revealed highly elevated levels of ABCB1 (P-gp, a drug efflux transporter) mRNA in clones D, E, and F as compared to the low levels in the parental cells as shown in FIG. 6 of parental cells and clones A-F vs. ABCB1 mRNA expression (RPKM). It should be noted that in FIG. 6, levels are measured as the number of reads per kilobase, per million reads (RPKM). This is consistent with the effective BI-2536 concentration being actively reduced in these clones. Since clones D, E, and F likely share a common ancestor, it is improbable that the up-regulation of the ABCB 1 transporter occurred independently in these lines. Importantly, clones D, E, and F are less sensitive than the parental cell line to taxol (paclitaxel), a compound known to be transported by ABCB1 as shown in FIG. 11. This data indicates that the method of the present invention can reveal indirect drug resistance mechanisms, such as increased drug efflux, which is commonly seen in clinical resistance.

Three other clones had mutations in PLK1 and in these clones ABCB 1 levels were not increased, clones A, B, C as shown in FIG. 6 of parental cells and clones D-F versus normalized cell growth in 20 nM paclitaxel. Our analysis indicated there were no other mutations common within these clones, or with the other analyzed clones, which is consistent with the BI-2536-resistance conferring mutations in PLK1 arising independently.

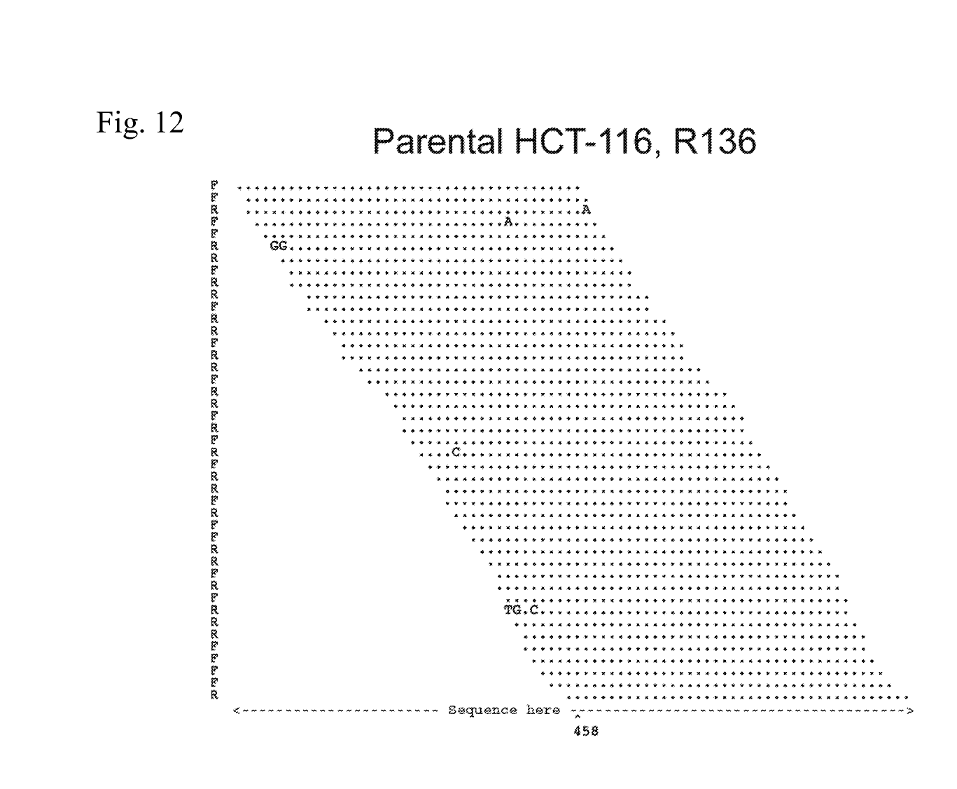

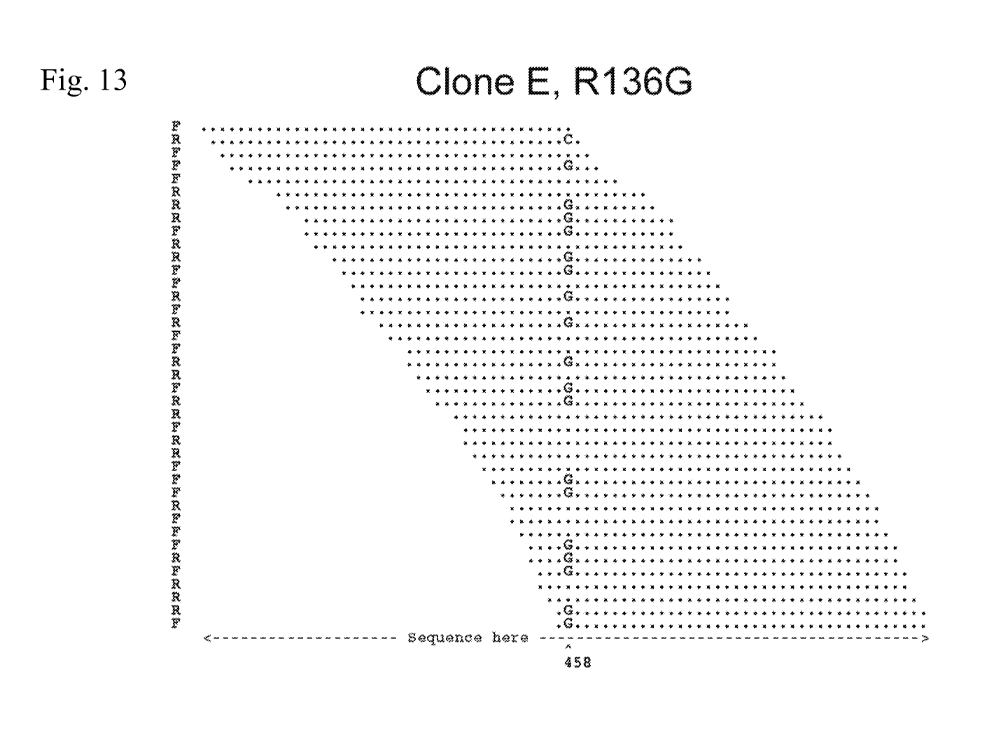

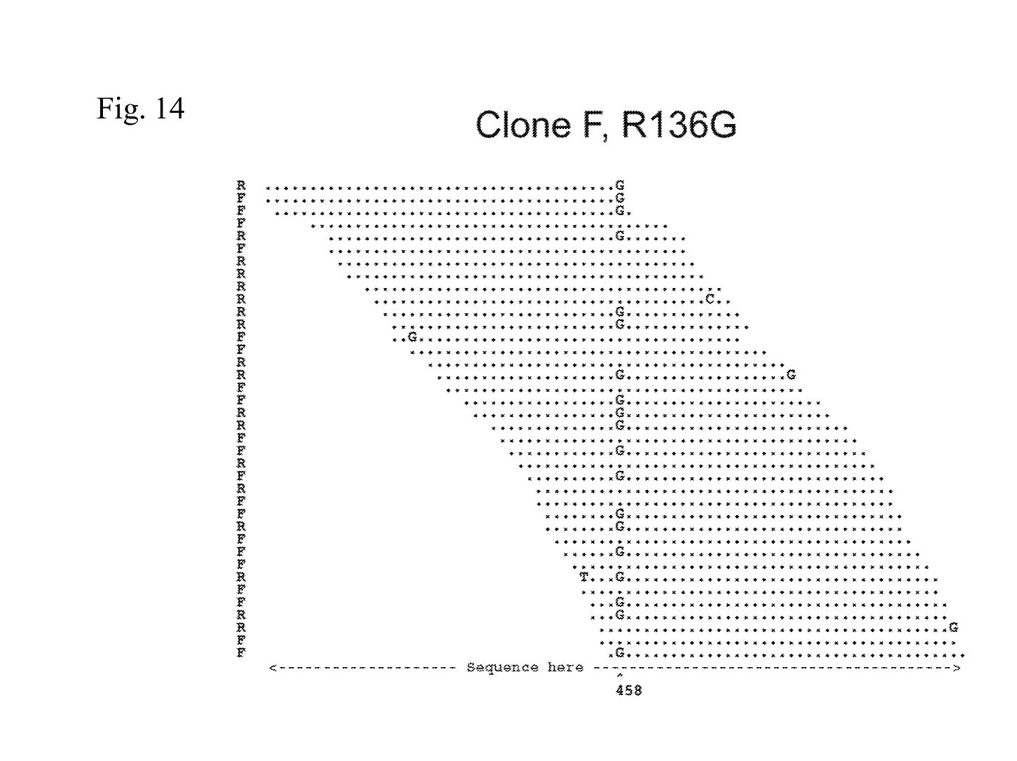

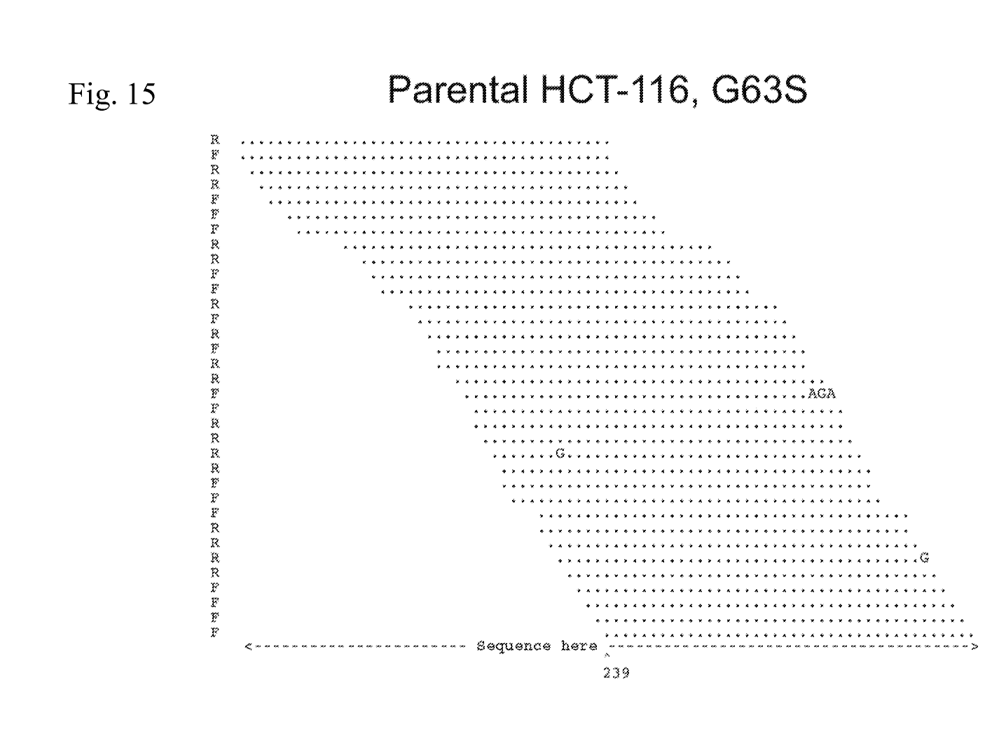

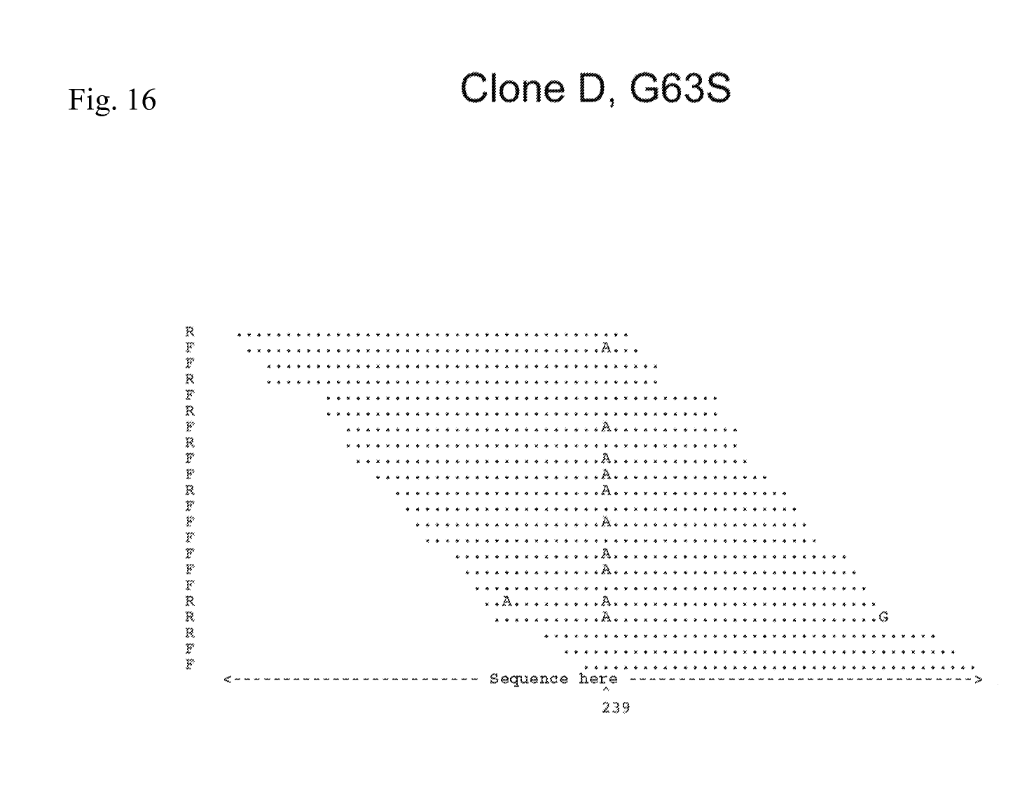

Of the genes identified by our analysis, PLK1 was the only one in which two distinct mutations were found, G63S and R136G. These mutations were not detected in the original HCT-116 cell population, despite high expression of PLK1 in these cells as shown in FIGS. 12-16, where the dots correspond to read nucleotides identical to the nucleotide in the reference genome hg18 and the direction with which reads mapped to the transcripts is indicated by F=forward and R=reverse. Furthermore, analysis of PLK1 mutations by RT-PCR and Sanger sequencing of the nine clones not subjected to RNA-seq indicated that mutations in the target is a common mechanism of BI-2536 resistance.

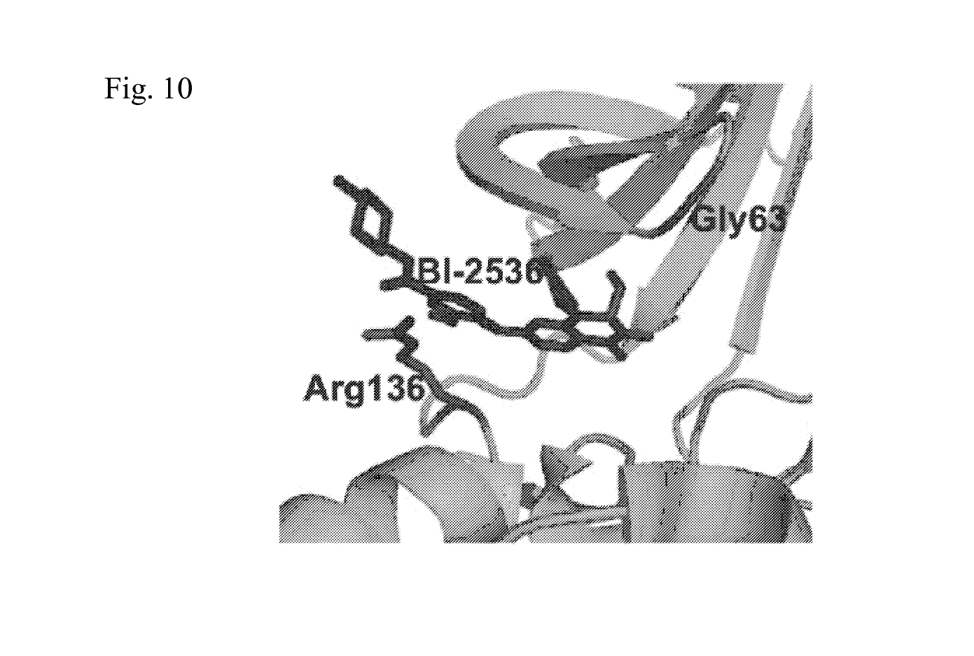

The two PLK1 mutations, G63S and R136G, map to the binding site of BI-2536 in the crystal structure of PLK1 as shown in FIG. 10. G63S, a mutation that has not previously been reported, likely occludes the binding of BI-2536 by replacing a small amino acid with one that has a bulkier side-chain. R136G has been shown to suppress BI-2536 inhibition (IC₅₀ increase of ˜3 fold, in vitro kinase assays), most likely due to loss of favorable interactions of the drug with the arginine side-chain. These mutations must not dramatically alter kinase activity, as their presence rescues cell growth when the wildtype copy of PLK1 is inhibited by BI-2536.

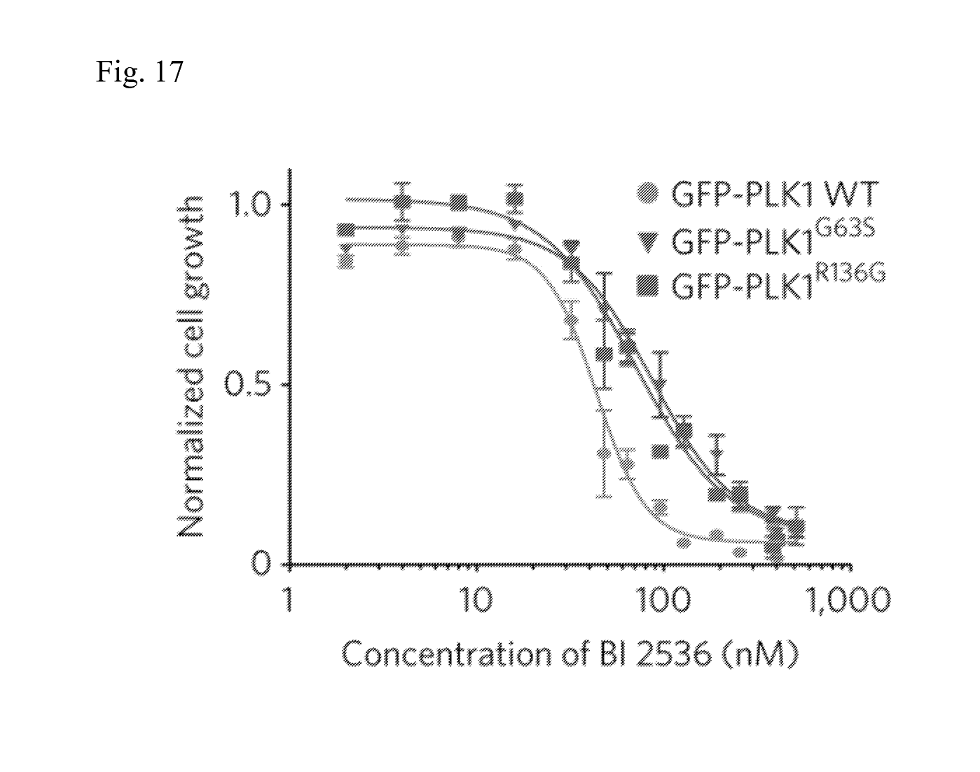

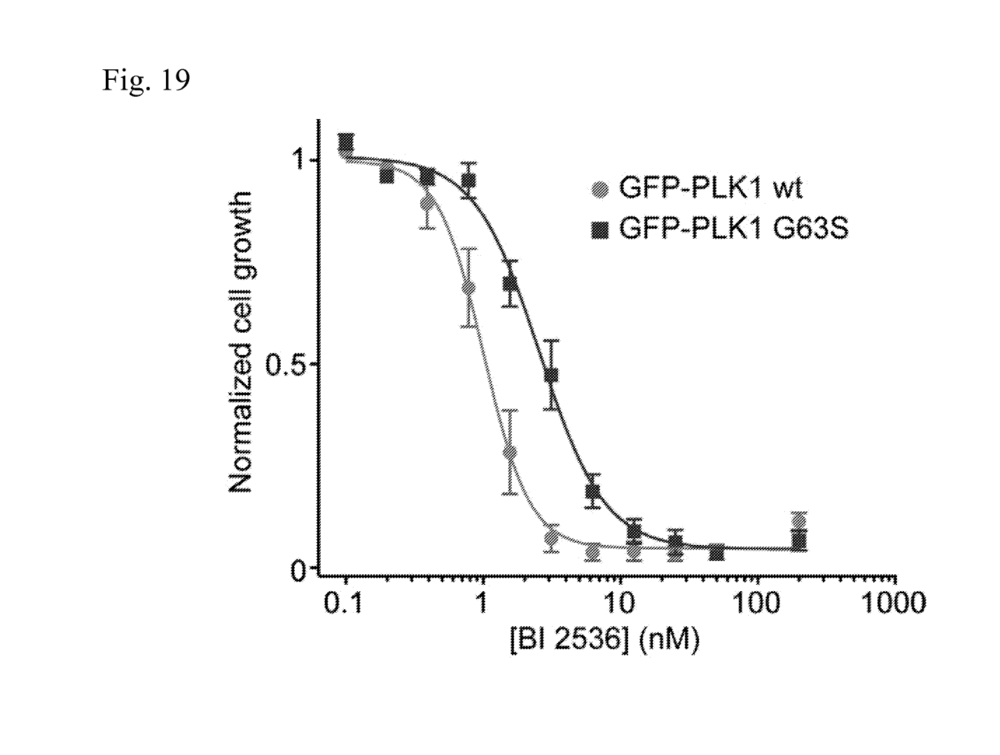

We next examined whether these mutations in PLK1 are sufficient for conferring resistance to BI-2536. This test is crucial as mutations in other genes (observed only once) were present in each clone. We expressed these mutations (PLK1 G63S and PLK1 R136G) in the full-length protein as a GFP-tagged construct in an independent cell line (hTERT-RPE1), which was known to be sensitive to BI-2536. We found that expression of each mutant construct suppressed BI-2536 toxicity in hTERT-RPE1 cells, stably expressing GFP-PLK1 WT, GFP-PLK1^{G36S} or GFP-PLK1^{R136G} as shown in FIG. 17, where the median lethal dose (LD₅₀s) measured for each transfected cell line was LD₅₀s: 44±5 nM for GFP-PLK1 WT, LD₅₀s: 83±9 nM for GFP-PLK1^{G36S}, and LD₅₀s: 76±8 nM for GFP-PLK1^{R136G}, with n=6, mean±s.e.m. P<0.01 for the two-tailed paired t-test. The effects of BI-2536 exposure on HeLa cells transfected with GFP-PLK1 wt and GFP-PLK1^{G63S} was also carried out as shown in FIG. 18, where the median lethal dose (LD₅₀s) measured for transfected HeLa cells are 1.0±0.2 nM GFP-PLK1 wt, and 2.7±0.3 nM for GFP-PLK1 G63S, with p<0.05, n=4, mean±s.e.m.

Therefore, there is evidence that BI-2536 is active against two other members of the Polo-like kinase family (PLK2,3) in vitro. Of these three potential targets, our data indicate PLK1 is the most likely physiologically relevant target of BI-2536. Thus our genomics analysis, can lead to a drug's target and meet the ‘gold standard.’

### Example 3

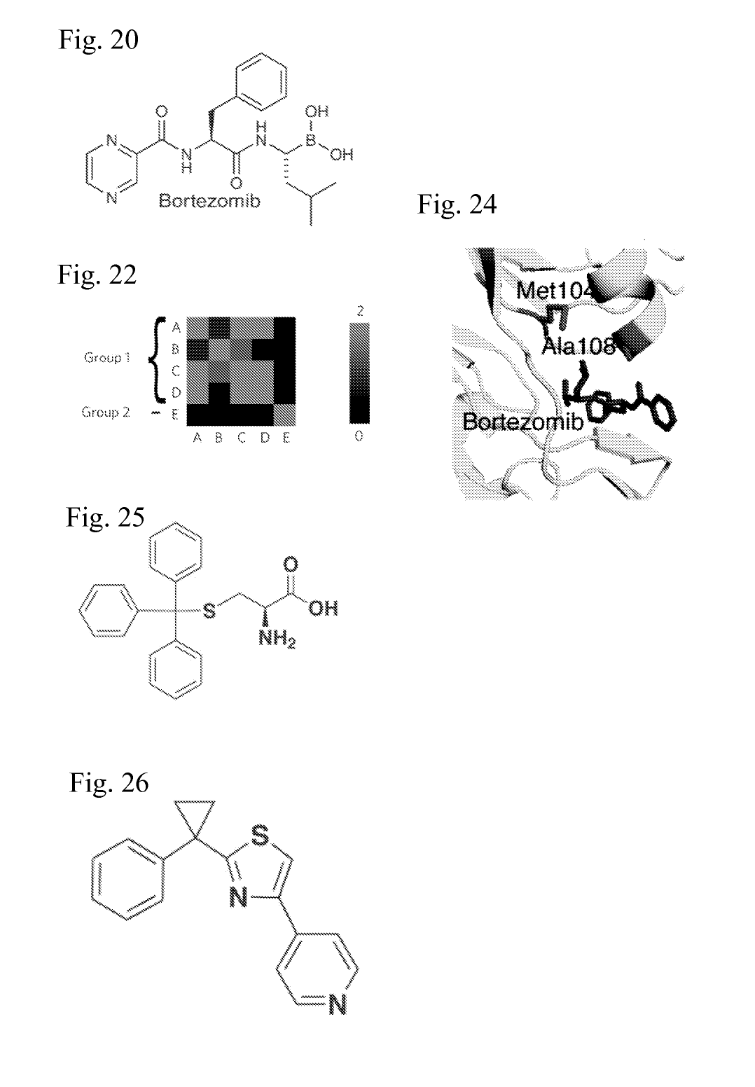

In a second example, to another drug, Bortezomib, which inhibits the proteasome by targeting the proteasomal subunit PSMB5 and is used clinically to treat multiple myeloma and mantle cell lymphoma was used instead of BI-2536. The specifics of the exact methodology used is discussed below under the title “Methods”. The structure of proteasome inhibitor bortezomib is shown in FIG. 20.

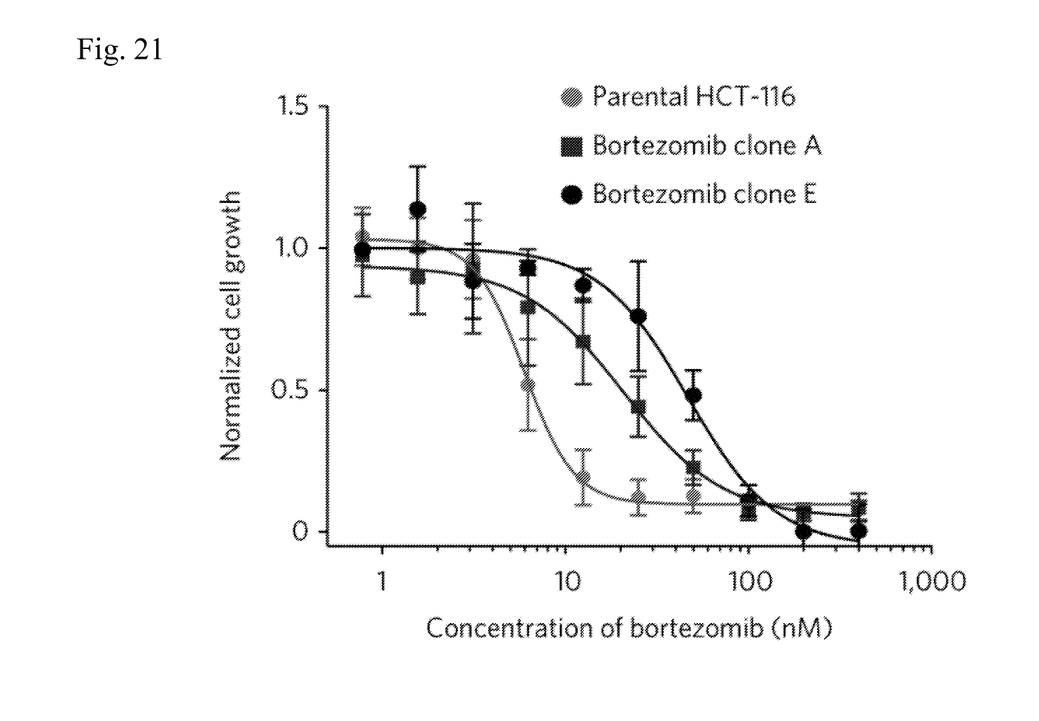

Nineteen clones were isolated from HCT-116 cells grown in the presence of bortezomib (8-12 nM; LD₅₀: 6.3±0.9 nm). Five clones with reduced bortezomib sensitivity, with LD₅₀ values 2.4-6.5 fold higher as shown in Table 4 and in FIG. 21 of concentration of bortezomib (nM) versus normal cell growth. The five clones were processed for transcriptome sequencing.

TABLE 4
mean ± s.e.m., n = 3)

| Clone | LD₅₀ (nM) |
|---|---|
| Parental | 6.3 ± 0.9 |
| A | 19.4 ± 6.7 |
| B | 15.3 ± 6.2 |
| C | 17.8 ± 6.0 |
| D | 17.2 ± 5.4 |
| E | 41.5 ± 9.5 |

In each clone, 15-28 single nucleotide variations were identified. Clustering analysis grouped clones A, B, C, and D together (group 1), whereas clone E was independent (group 2) as shown in FIG. 22. Five genes were mutated in both bortezomib-resistant groups, and the only gene with two distinct mutations, M104V and A108T, was gene PSMB5, encoding the known target of bortezomib as shown in Table 5 below.

TABLE 5

| | |
|---|---|
| | Bortezomib |
| Amino Acid | Group |

| Gene | Mutation | 1 | 2 |
|---|---|---|---|
| PSMB5 | A108T | X | |
| PSMB5 | M104V | | X |
| PPP1R10 | R58H | X | X |
| CDK5RAP3 | V21G | X | X |
| ETHE1 | S19A | X | X |
| ID1 | S8R | X | X |

If the drug target was unknown, all five gene products would have had to be examined as potential targets. However, the existence of two distinct resistance mutations made PSMB5 the highest priority for further analysis.

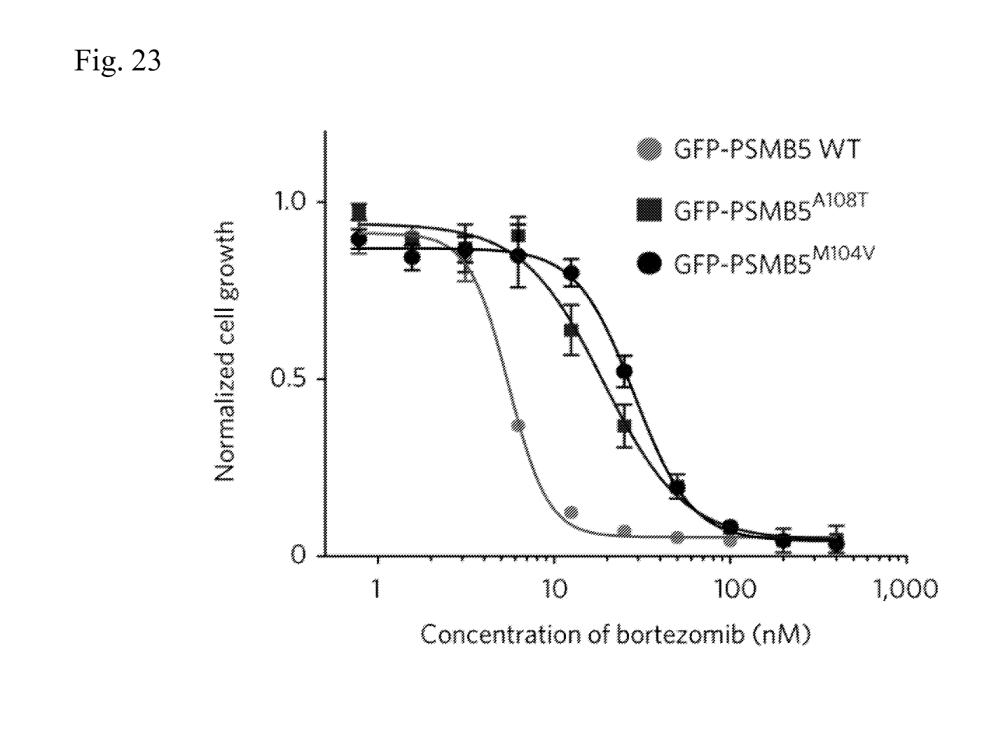

It was found that stable expression of GFP-tagged PSMB5, carrying either the M104V or A INT mutation, suppressed bortezomib sensitivity in an independent cell line hTERT-RPE1 as show in FIG. 23. This result was consistent with reports that A108T confers bortezomib resistance. As both mutations in PSMB5 map to the drug's binding site, we inferred that they directly suppress drug interactions. As shown in FIG. 24, which includes the structure of bortezomib with Pre2, a yeast homolog of PSMB5, amino acids Met104 and Ala108 in Pre2, a subunit of the 20S proteasome, are proximal to the bortezomib binding site (pdb: 2F16²). Our data indicates that the method of the present invention can efficiently lead to resistance mechanisms that include a drug's direct target.

Based on the examples above, the method of the present invention is effective. Resistance via mutations in a drug's direct target must occur at high frequency in drug-resistant clones. To examine this, the PLK1 gene in each of the nine Bi-2536-resistant clones that we had not processed by RNA-seq was sequenced. PLK1 was mutated in ˜45% of these clones as shown in Table 6 below, with RT-PCT and Sanger sequencing.

TABLE 6

| Clone | PLK1 Mutations |
|---|---|
| 1 | None |
| 2 | None |
| 3 | R136G |
| 4 | R136G |
| 5 | R136G |
| 6 | None |
| 7 | None |
| 8 | None |
| 9 | R136G |

The two kinesin-5 inhibitors, S-trityl-L-cysteine (STLC), the structure of which is shown in FIG. 25, which is known to be selective, and 4-(2-(1-phenylcyclopropyl)thiazol-4-yl)pyridine (PCTP), the structure of which is shown in FIG. 25, which has been shown to inhibit other related motor proteins in vitro were analyzed. Kinesin-5 mutations were found in ˜30% of the STLC-resistant clones as shown in Table 7 and in ˜15% of PCTP-resistant clones as shown in Table 8.

TABLE 7

| Clone | Kinesin-5 Mutation |
|---|---|
| 1 | E123K |
| 2 | None |
| 3 | None |
| 4 | None |
| 5 | None |
| 6 | None |
| 7 | A334V |
| 8 | None |
| 9 | H354R |
| 10 | None |
| 11 | None |
| 12 | None |
| 13 | A103V |
| 14 | None |

TABLE 8

| Clone | Kinesin-5 Mutation |
|---|---|
| 1 | None |
| 2 | None |
| 3 | None |
| 4 | None |
| 5 | None |
| 6 | None |
| 7 | None |
| 8 | None |
| 9 | None |
| 10 | S269N |
| 11 | S269N |
| 12 | Y104H |
| 13 | None |
| 14 | None |
| 15 | None |
| 16 | None |
| 17 | None |
| 18 | None |
| 19 | None |
| 20 | None |
| 21 | None |
| 22 | None |

This data indicates that resistance in a drug's direct target occurs at a high frequency when a drug has one major physiological target (as is the case for STLC, BI-2536 and bortezomib). When a drug has multiple targets (for example, PCTP), it is likely that resistance in a single target will be less frequent and that our approach may be limited, as a greater number of clones would have to be sequenced to identify mutations common in more than one clone.

We have described a general and unbiased method that can identify drug-resistance conferring mutations in human cells. This method consists of identifying genes harboring mutations absent (or present below detectable levels) in an original parental cell population and present in expanded drug-resistant clones. By using transcriptome sequencing, bioinformatic analyses, and isolating multiple drug-resistant clones, we overcame obstacles such as the large size of the human genome and the high levels of genetic heterogeneity that can be found in human cell lines (e.g. cancer cells). Importantly, the use of RNA-seq provides mRNA transcript level information, which can be crucial to the discovery of some mechanisms of drug resistance, such as up-regulation of drug efflux pumps.

In addition to identifying resistance mechanisms, the method of the present invention can define proteins that are a drug's direct target. In order to definitively identify the target of a given drug, our approach is followed by cytological and biochemical characterizations of the drug's mechanism of action. Once this characterization has been completed, the drug target identification meets the ‘gold standard’. Furthermore, unlike many other target identification approaches, the method of the present invention does not rely on chemical modifications of the drug of interest. This can be important when small changes in a drug's chemical composition can alter its mechanism of action.

The method of the present invention may be applied to any cells that can be grown in culture, enabling cell-type specific analyses. Such analysis may be particularly useful if a drug has unexpected toxicity in specific tissues. The method of the present invention is not limited to analyzing cytotoxic drugs and is applicable to non-toxic drugs using phenotypic or reporter-based read-outs to select clones (e.g. fluorescence changes) with reduced drug response from a heterogeneous starting population, without needing to select for cell growth. Furthermore, the method of the present invention is not limited to single nucleotide variations and insertions/deletions and can be used to report on all potential mechanisms of resistance. This is done by combining the method of the present invention with other genomic methods, such as exome capture, full genome sequencing, and bisulfate conversion followed by sequencing (to detect DNA methylation). In summary, the method of the present invention can reveal all the physiological on-targets of a drug in disease cells, unintended off-targets in healthy cells, and can reveal cellular mechanisms of drug resistance. These findings can impact chemical modifications of drugs to improve efficacy and limit toxicity. Furthermore, when unanticipated drug targets are found, new uses of the drugs can also be discovered.

In summary, the method of the present invention identifies the target of a drug in human cells by examining resistance mechanisms. The method involves isolating multiple-drug-resistant clones from genetically heterogeneous human cells. Clones with multidrug resistance can be excluded by testing for reduced sensitivity to unrelated compounds (for example, paclitaxel). The remaining clones are processed for transcriptome sequencing, along with the parental (untreated) cell population. Bioinformatics is used to find genes mutated in more than one independent clone. These genes are prioritized for further biochemical and cell biological analyses to identify the drug's direct target and indirect resistance mechanisms.

Methods

Molecular and Cell Biology

HCT-116 cells and clonal lines were cultured in McCoy's 5A medium (Invitrogen). hTERT-RPE1 cells were cultured in Dulbecco's Modified Eagle's Medium/F12 1:1 nutrient mix (Invitrogen), while HeLa and 293-Ampho cells were cultured in Dulbecco's Modified Eagle's Medium (Invitrogen). All cultures were supplemented with 10% FBS (Atlanta Biologicals) and penicillin-streptomycin (100 U/ml and 100 ug/ml, respectively, Invitrogen) and grown at 37° C. in a humidified chamber with 5% CO2. hTERT-RPE1, HeLa, and 293-Ampho cells were also supplemented with 1% MEM non-essential amino acids (Invitrogen) and 2 mM 1-glutamine (Invitrogen).

Human PLK1 (image clone ID#2822226, Open Biosystems) or human PSMB5 (image clone ID#4795732, Open Biosystems) was cloned into a pMSCVpuro vector (Clontech) with an N-terminal GFP-PreScission protease site compatible with the Gateway cloning system (Invitrogen). Site-directed mutagenesis to generate the PLK1 R136G and G63S mutations or the PSMB5 M104V and A108T mutations was performed using QuickChange (Stratagene) according to the manufacturer's instructions. DNA encoding the wildtype and mutant proteins was used to generate stable cell lines through retroviral infection. Retroviruses were packaged in 293-Ampho cells. hTERT-RPE1 and HeLa cells were infected by retrovirus with 4 μg/ml polybrene (Sigma) and selected by puromycin (Sigma). Transfection levels of the wildtype and mutant proteins in hTERTRPE1 cells were confirmed by Western blot.

Selection of Resistant Clones

BI-2536 (>99% pure) was purchased from Selleck chemicals. Bortezomib (99% pure) was purchased from LC Laboratories. S-trityl-L-cysteine (STLC, 97% pure) was purchased from Sigma. 4-(2-(1-phenylcyclopropyl)thiazol-4-yl)pyridine (PCTP, >95% pure) was synthesized.

Resistant clones were generated by plating 0.5-1.0×10₆ HCT-116 cells into 10 cm culture dishes with media containing 10 nM BI-2536, 1 μM STLC, 8-12 μM PCTP, or 8-12 nM bortezomib. Media with compound was exchanged every three days for two-four weeks. Most cells did not survive, but a few per plate grew into colonies (less than 20 colonies were found on each plate). Colonies were picked by ring cloning and transferred to a new plate where they were maintained in media containing drug at the same concentration as the selections.

Cell Proliferation Assays and Calculation of LD₅₀ Values

In order to quantify cell growth in the presence of drug, cells (1000 in 100 μl of media per well) were plated in a flat-bottomed 96-well plate and treated the next day with various concentrations of the appropriate compound, in duplicate. After three days, cell proliferation was determined using a WST1 assay (Millipore) according to the manufacturer's instructions. Normalized cell proliferation was calculated as the change in the number of cells at each concentration compared to control wells. The assay was repeated three-six independent times. Normalized cell proliferation values were plotted and LD₅₀ values were obtained by curve fitting with Prism. A two-tailed paired t-test was used to determine statistical significance.

Normalized cell proliferation values were plotted and LD₅₀ values were obtained by curve fitting using the equation:

y=M1+(M2−M1)/(1+(x/M3))

where:
M1 is the minimum y value,
M2 is the maximum y value, and
M3 is the LD₅₀.

RNA Purification and RT-PCR

Total RNA was isolated from cells using the RNeasy Mini Kit (Qiagen) according to the manufacturer's instructions. Full-length PLK1 or kinesin-5 cDNA was synthesized and amplified from total RNA using PLK1 or kinesin-5 specific primers and the SuperScript III One-Step RT-PCR System (Invitrogen).

RNA-Seq Library Construction

Following isolation, total RNA integrity was checked using an Agilent Technologies 2100 Bioanalyzer and an RNA Integrity Number greater than 8 was required for further processing. Library construction was performed according to standard Illumina protocols with Illumina reagents. Briefly, mRNA was purified from total RNA using magnetic beads: 5-100 ng of total RNA were heated to disrupt the secondary structures and then added to pre-prepared Sera-mag Magnetic Oligo(dT) Beads. After washing, 10 mM Tris-HCl was added to the beads, the samples were heated, and mRNA was eluted. The mRNA was then fragmented using divalent cations under elevated temperatures. The cleaved RNA fragments were copied into first strand cDNA using reverse transcriptase and random primers. mRNA was removed by RNaseH and a replacement strand was synthesized to generate double-strand cDNA. The overhangs resulting from fragmentation were converted into blunt ends by T4 DNA polymerase and DNA polymerase I Klenow fragment. An ‘A’ base is added to the 3′ end of the blunt phosphorylated DNA fragments to prepare them for ligation to the adapters, which have a single ‘T’ base overhang at their 3′ end. Illumina adapters were ligated to the ends of the DNA fragments, preparing them to be hybridized to a flow cell. Ligation reaction products were purified on an agarose gel. A 200±25 by size-range of templates was selected for downstream enrichment. The cDNA fragments with adapters on both ends were amplified by PCR with primers complementary to the adapters. Size, purity and concentration of the library were checked on an Invitrogen Qubit Fluorometer using the Quant-IT dsDNA HS Assay Kit and on an Agilent Technologies 2100 Bioanalyzer using their High Sensitivity DNA Kit.

High-Throughput Sequencing

Sequencing was performed using both Illumina GAIIx (for BI-2536 clones) and HiSeq2000 (for bortezomib clones) machines. For GAIIx, the protocols for the Illumina Single-Read Cluster Generation Kit were used for cluster generation on the Cluster Station. The targeted samples were diluted to ten nanomoles and denatured with sodium hydroxide. Ten picomoles of each target-enriched sample and control was loaded into separate lanes of the same flow cell, hybridized onto the flow cell, and isothermally amplified. After linearization, blocking, and primer hybridization, sequencing was performed for 40 cycles on the Illumina 36 Cycle Sequencing Kit v4 with version 7.0 sequencing protocols. Raw image data was converted into base calls using the Illumina pipeline v1.6 with default parameters. Rigorous quality control was performed using data from reports generated by the Illumina pipeline. For HiSeq2000, a similar protocol was used, and some of the clones were sequenced on the same lane (3-plex). After quantifying and checking the size and purity of the product, multiplexed DNA libraries were normalized to 10 nM and then sample libraries were pooled together in equal volumes. 7 pM of each pooled DNA library templates was amplified on Illumina cBot instrument involving immobilization and 3′ extension, bridge amplification, linearization and hybridization, then sequenced on the Illumina HiSeq2000 sequencer using 51 cycles.

Alignment of RNA-Seq Reads

RNA-seq reads were aligned to RefSeq transcript sequences downloaded from the UCSC Genome Browser in June 2010, using the BWA program4 with default parameters. Out of 25-100 million reads obtained in each run, 74-83% could be mapped to RefSeq transcripts. Clonal reads, i.e. multiple reads mapping at the same position and same orientation in a transcript, were collapsed into a single read. Following mapping to RefSeq transcripts to identify reads mapping to exons and across known exon junctions, all mapped reads were remapped to the reference human genome using custom programs and based on the June 2010 RefSeq gene annotation.

Overall Bioinformatics Strategy

The following strategy was used to determine which genetic variants increased their relative abundance in the expanded clones compared to the original cell population. First, single nucleotide variations (SNVs) and insertions/deletions (indels) in the sequenced mRNAs of each clone were identified using the statistical approaches described below. Next, the relative abundance of these variants was compared to the relative abundance in the same variants (at the same location) in the original cell population. Only variants whose relative abundance had increased significantly (after correction for multiple hypothesis testing) in the expanded clone were retained. Finally, variants with unlikely functional impact, e.g. synonymous variants and variants in 5′UTRs and 3′UTRs were filtered out.

Single Nucleotide Variant (SNV) Detection

For each nucleotide in RefSeq transcripts, we calculated the number of overlapping reads (denoted as n) and determined how many of these reads showed a mismatch compared to the reference hg18 human genome sequence. The number of reads with a mismatch is denoted as k. We also recorded the position of the n-k matches and k mismatches within the 40 or 51 bp-long reads. We then determined the probability of observing k mismatches or more by chance, given the location of these mismatches within the reads and the overall error rate observed at each read position. Because most mismatches are expected to be sequencing errors (as opposed to biological variation), the error rate at position i, denoted as pᵢ, is the number of mismatches occurring at position i in the entire sequencing experiment divided by the total number of mapped reads. Under these conditions, the probability of observing k mismatches by chance is determined by the Poisson-Binomial distribution:

$$P ( {S}_{Z} = k ) = \{ {\prod }_{i = 1}^{n} ( 1 - {p}_{i} ) \} {\sum }_{{i}_{1} < … < {i}_{k}} {w}_{{i}_{1}} … {w}_{{i}_{k}}$$

where wᵢ=pᵢ/(1−pᵢ) for 1=1 . . . n. The Poisson-Binomial distribution describes the distribution of sums of Bernouilli variables S_{z}=Z₁+ . . . Zₙ, i.e. where Z can take 0 or 1 values, with p(1)=pᵢ. Modeling error rates at distinct read positions is important because in Illumina sequencing, the number of sequencing errors is often high at the first position in reads and typically increases with the distance from the beginning of reads. Using the Poisson-Binomial ensures that mismatches occurring in read regions with high sequencing error rates get lower weight than mismatches occurring in regions with low error rates. Poissonbinomial p-values, i.e. P(S_{z}≧k), were calculated using the algorithm. P-values were only calculated for transcript positions with sufficient number of reads, i.e n≧4 in this study. To take into account multiple hypothesis testing, p values were then adjusted using the Benjamini-Hochberg approach and a false discovery rate of 1% was used for SNV calling. A post-calling filter was applied to heterozygous variants, which compares Illumina quality scores (QS) of the variant nucleotides to the reference ones at the variant position; variants with variant nucleotide QS that were significantly lower (p<0.01, Wilcoxon test) than reference nucleotide QS were eliminated.

Insertion/Deletion (Indel) Detection

We used a similar but slightly simpler approach to call indels, by assuming that the indel error rate is uniform along read lengths (since we did not observe a very strong position-specific indel rate). Here too, we assume that most insertions and deletions are sequencing errors and calculate the probability of observing k insertions (or k deletions) out of n reads mapping at a given transcript position, given the overall indel error rate p (distinct for deletions and insertions). Under these conditions, the probability of observing k insertions (or k deletions) follows a binomial distribution:

$$P ( X \geq k ) = {\sum }_{k}^{n} ( \begin{matrix}n \\ k\end{matrix} ) {{p}^{k} ( 1 - p )}^{n - k}$$

As for SNVs, p-values were only calculated for transcript positions with n 4 reads. These p-values were then adjusted using the Benjamini-Hochberg approach and a false discovery rate of 1% was used for indel calling. The analysis was run twice, once to detect insertions and once to detect deletions.

Comparing Variant Abundances

To compare relative variant abundances between an expanded clone and the parental cell population and to detect variants whose relative abundance is increased in the expanding clones, we used the hypergeometric distribution. The probability that the number of reads harboring a given variant in the expanded clone (denoted as k_{clone}) out of n_{clone} reads is identical to the number of reads harboring this variant in the parental cell population (denoted as kₚₐᵣₑₙₜₐₗ) out of nₚₐᵣₑₙₜₐₗ reads is given by:

$$P ( X = {k}_{clone} ) = \frac{( \begin{matrix}{n}_{clone} \\ {k}_{clone}\end{matrix} ) ( \begin{matrix}{n}_{parental} \\ {k}_{parental}\end{matrix} )}{( \begin{matrix}{n}_{clone} + {n}_{parental} \\ {k}_{clone} + {k}_{parental}\end{matrix} )}$$

In this context, the probability (p-value) that k_{clone}/n_{clone} reads is greater than kₚₐᵣₑₙₜₐₗ/nₚₐᵣₑₙₜₐₗ is given by:

$$P ( X \geq {k}_{clone} ) = {\sum }_{x = {k}_{clone}}^{{n}_{clone}} P ( X = x )$$

Importantly, this statistical analysis requires that genes harboring variants in the clone be also expressed in the parental population; a variant with high abundance in a clone, but in a gene that is not expressed in the parental population, would not be detected (because lack of expression means that we don't know whether the mutation is present or not).

To limit the number of hypotheses to test, we only apply this test to variants detected in the expanded clones using the variant calling procedures described above (which define variants as mismatches compared to the hg18 reference human genome). Hypergeometric p-values were adjusted using the Benjamini-Hochberg approach and a false discovery rate of 0.5% was applied to determine variants with increased abundance.

Biological Filtering of Detected Variants

Using RefSeq gene annotation (downloaded from UCSC Genome Browser in June 2010), we determined, through custom scripts and programs, where all variants were located (coding sequence, 5′UTR, and 3′UTR). Variants in 5′UTRs and 3′UTRs were filtered out as they are unlikely to contribute to drug resistance. Additional custom programs were used to determine whether SNVs in coding sequences were synonymous (no amino acid change), missense (amino acid change) or nonsense (premature stop codon introduced). The position in the protein sequences where missense and nonsense mutations occur was also determined Synonymous variants were excluded from the analysis as they are also unlikely to contribute to drug resistance.

RNA-Seq Transcript Abundance Estimation

Transcript abundances from the RNA-seq data generated for drug-resistant clones and the original HCT-116 cell population were estimated using the RPKM approaches. Briefly, after reads were mapped to RefSeq transcripts as described above, the total number of reads mapping to each transcript was determined. When a read mapped to more than one transcript, the read was randomly associated to one of the transcripts. For each transcript, the number of reads mapping to it was divided by the length of the transcript (in nucleotides) and multiplied by 1,000. Then, in order to compensate for the slightly unequal number of RNA-seq reads obtained for each clone, the length-normalized read counts were multiplied by a factor equal to 1 million divided by the number of mapped reads obtained in the RNA-seq run.

Graph-Based Clone Clustering and Merging

To determine whether a set of clones shared a significant number of single nucleotide variants and are therefore not genetically independent, we first created a master list of all variants found in all clones to compare. We then compared pairs of clones using the hypergeometric distribution. In a given pair, assuming that clone 1 has s₁ variants and clone 2 has s₂ variants, that they share i variants and that the total number of variants in the set of clones is N, the dissimilarity between the two clones was calculated using:

$$P ( X \geq i ) = {\sum }_{x = i}^{min ( {s}_{1} , {s}_{2} )} \frac{( \begin{matrix}{s}_{1} \\ x\end{matrix} ) ( \begin{matrix}N - {s}_{1} \\ {s}_{2} - x\end{matrix} )}{( \begin{matrix}N \\ {s}_{2}\end{matrix} )}$$

For this application, two clones were considered similar if P(x≧i)<0.1. A simple graph where nodes are clones connected by an edge if the two clones are similar was then created. A simple depth-first search algorithm was then used to find connected components within this graph. Clones within each component were merged, such that any mutation observed in a component clone was inherited by the group. The negative logarithm base 10 of the hypergeometric p-values was used to draw the heatmaps in FIGS. 7 and 22.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of identification of drug targets and drug resistance mechanisms in human cells of a drug comprising:
a) generating at least one drug-resistant sample, comprising at least one drug-resistant cell, wherein the at least one drug-resistant cell in the sample is substantially resistant to the drug and wherein the at least one drug-resistant sample is obtained in vitro from an immortalized normal cell line, a transformed cell line or a disease cell line or the at least one drug-resistant sample is obtained in vivo from immortalized normal tissue or disease tissue;
b) generating at least one drug-sensitive sample, comprising of at least one drug-sensitive cell, wherein the at least one drug-sensitive cell in the sample is sensitive to the drug and wherein the at least one drug-sensitive sample is obtained in vitro from an immortalized normal cell line, a transformed cell line or a disease cell line or the at least one drug-resistant sample is obtained in vivo from immortalized normal tissue or disease tissue
c) analyzing substantial portions of the genome and/or transcriptome of the least one drug-resistant sample to obtain sequencing data using one of the following methods from the group consisting essentially of: exomic sequencing, genomic sequencing, transcriptome sequencing, epigenomic sequencing, and high-throughput sequencing;
d) analyzing substantial portions of the genome and/or transcriptome of the least one drug-sensitive sample to obtain sequencing data using one of the following methods from the group consisting essentially of: exomic sequencing, genomic sequencing, transcriptome sequencing, epigenomic sequencing, and high-throughput sequencing;
e) detecting substantially all alterations in the at least drug-resistant sample by comparing the sequencing data for the at least one drug-resistant sample to sequencing data of the at least one drug-sensitive sample;
f) deriving a resistance signature by merging the alterations and genes affected by the alterations from the at least one resistant sample and substantially similar resistant cells of the at least one resistant sample with the filtered and identified data generated from the detection of alterations of the at least one resistant sample to obtain a drug resistance signature of at least one recurrently altered gene that has drug resistance across multiple independent resistant cells of the at least one resistant sample; and
g) performing analysis of the drug resistance signature of at least one recurrently altered gene using bioinformatic tools and cellular biology methods to determine if alteration of the at least one gene of the drug resistance signature is sufficient to confer at least partial resistance to cells or tissues against the drug.

2. The method of claim 1, wherein the at least one drug-resistant sample has substantially reduced sensitivity to the drug.

3. The method of claim 1, wherein the at least one resistant sample to the drug is derived by growing cells in vitro at doses close to, but lower than a measured lethal dose of the drug.

4. The method of claim 1, wherein the at least one resistant sample to the drug is derived by selecting cells that express a marker, reporter gene or phenotype that indicates that a cell is resistant to the drug.

5. The method of claim 1, wherein the at least one resistant sample to the drug is obtained by injecting at least one disease cell from a cell line into at least one animal, the at least one disease cell may divide within the at least one animal, the at least one animal is treated with the drug continuously or using multiple on/off treatment cycles so as to select for samples with reduced sensitivity to the drug, and the at least one resistant sample is collected from the at least one animal.

6. The method of claim 1, wherein the at least one resistant sample to the drug is derived by treating at least one animal with the drug, where the at least one animal may be genetically engineered or not and collecting the at least one resistant sample from the at least one animal.

7. The method of claim 1, wherein the at least one resistant sample to the drug is derived from at least one human treated with the drug and collecting the at least one resistant cell from the human.

8. The method of claim 1, wherein the at least one sensitive sample to the drug is derived by selecting cells that express a marker, reporter gene or phenotype that indicates that a cell is sensitive to the drug.

9. The method of claim 1, wherein the at least one sensitive sample to the drug is obtained by injecting at least one disease cell from a cell line into at least one animal, the at least one disease cell may divide within the at least one animal, the at least one animal is treated with the drug continuously or using multiple on/off treatment cycles so as to select for samples with sensitivity to the drug, and the at least one sensitive sample is collected from the at least one animal.

10. The method of claim 1, wherein the at least one sensitive sample to the drug is derived by treating at least one animal with the drug, where the at least one animal may be genetically engineered or not and collecting the at least one sensitive sample from the at least one animal.

11. The method of claim 1, wherein the at least one sensitive sample to the drug is derived from at least one human treated with the drug and collecting the at least one sensitive cell from the human.

12. The method of claim 1, wherein the at least one drug-sensitive sample is obtained from cells with the substantially similar genetic background as the at least one drug-resistant sample, and collected in vitro or in vivo.

13. The method of claim 1, wherein the at least one drug-resistant sample that is resistant due to expression of multidrug efflux pumps can be identified by treating the resistant sample with a drug known to be pumped out by the multidrug efflux pumps.

14. The method of claim 1, wherein the alterations are single nucleotide variations and indels and substantially all single nucleotide variations and indels in the at least one drug-resistant sample are detected using the steps comprising: identifying relevant single nucleotide variations and indels within the sequencing data of the at least one resistant sample and detecting the single nucleotide variations and indels from the at least one resistant cell with substantially increased abundance within the sequencing data of the at least one resistant sample compared to the at least one drug-sensitive sample.

15. The method of claim 1, wherein the alterations are changes in transcription level of a gene and substantially all changes in transcription level in the at least one drug-resistant sample are detected using the steps comprising: quantifying transcription levels by determining steady-state mRNA levels of substantially all genes from the sequencing data in the at least one drug-resistant sample and the at least one drug-sensitive sample and identifying substantially all genes whose transcription level is substantially different in the at least one drug-resistant sample compared to the at least one drug-sensitive sample.

16. The method of claim 1, wherein the alterations are gene fusions and substantially all gene fusions in the at least one drug-resistant sample are detected by finding instances of gene fusion, in which parts of two or more genes genetically recombine into a new gene with different or additional regulatory regions, in the sequencing data of the at least one drug-resistant sample and in the sequencing data of the at least one drug-sensitive sample and identifying gene fusions found in the at least one drug-resistant sample at a substantially higher abundance than in the at least one drug-sensitive sample.

17. The method of claim 1, wherein the alterations are copy number variations and substantially all copy number variations in the at least one drug-resistant sample are detected by detecting copy number variations, in which alterations of DNA results in an abnormal number of copies of one or more sections of DNA, comcomittant or not with loss of heterozigosity, in the sequencing data of the at least one drug-resistant sample and in the sequencing data of the at least one drug-sensitive sample and identifying regions with substantially different copy number in the at least one drug-resistant sample compared to the at least one drug-sensitive sample

18. The method of claim 1, wherein the at least one drug-resistant samples with the at least one drug-resistant cells with substantially similar alterations are identified by quantifying the similarity between the patterns of alterations and groups of drug-resistant cells and other drug-resistant samples with substantially similar alterations are merged into a single drug-resistant sample by determining a union of all alterations found in the drug-resistant samples and drug-resistant cells.

19. The method of claim 1, wherein the alterations and genes affected by the alterations from at least two drug-resistant samples resistant to the same drug are merged, sorted by frequency across the at least two drug-resistant samples, with recurrent or most frequent alterations prioritized, such that alterations directly related to substantial drug resistance are prioritized over ‘passenger’ alterations that do not contribute to the changes in drug sensitivity in the drug-resistant samples.

20. A method of identification of drug targets and drug resistance mechanisms in human cells of a drug using substantial portions of the genome and/or transcriptome of at least one drug-resistant sample to identify substantially all alterations in the at least one resistant sample, the method further comprising the steps of:
deriving a resistance signature by merging data derived from substantially similar drug-resistant samples with reduced drug sensitivity to the drug and merging the alterations obtained from the substantially similar drug-resistant samples to obtain a drug resistance signature of at least one recurrently altered gene and its alterations that has drug resistance across the drug-resistant samples and sorting the genes and alterations by how frequently the genes and alterations were independently obtained from the substantially similar drug-resistant samples and prioritizing the genes and alterations that are most frequently found;
analyzing the drug resistance signature of at least one recurrently altered gene using bioinformatic tools and/or cellular biology methods to determine if alteration of the at least one gene of the drug resistance signature is sufficient to confer at least partial resistance to cells against the drug; and
identifying at least one drug target or at least one drug mechanism from the drug resistance signature of a drug that is sufficient to confer at least partial resistance to cells and/or tissues against the drug.

21. The method of claim 20, wherein the analysis of the drug resistance signature of at least one recurrently altered gene includes mapping point mutations and indels onto a three dimensional protein structure to examine how the mutation interferes with biochemical activity.

22. The method of claim 20, wherein the analysis of the drug resistance signature of at least one recurrently altered gene for genes in which mutations are identified comprises stably expressing an allele in the cell line using retroviral based systems.

23. The method of claim 20, wherein the analysis of the drug resistance signature of at least one recurrently altered gene for genes over-expressed comprises RNAi-mediated knockdown in isolated and expanded drug-resistant clones.

24. The method of claim 20, wherein analysis of the drug resistance signature of at least one recurrently altered gene for an observed mutation in the drug's anticipated target comprises testing if the observed mutation can alter drug-target interaction through in vitro biochemical activity assays.

25. The method of claim 20, wherein the analysis of the drug resistance signature of at least one recurrently altered gene for an observed mutation in the drug's predicted direct target comprises testing if the observed mutation can alter drug-target interaction through binding.

26. The method of claim 20, wherein the analysis of the drug resistance signature of at least one recurrently altered gene to analyze interactions of a drug with a potential target comprises generating analogs for immobilization for chemical synthesis.

27. The method of claim 20, wherein the drug resistance signature is compared to genomic data for a human patient to identify whether cells of the patient will have drug resistance found in the drug resistance signature and providing data to influence drug usage for the patient.

28. The method of claim 20, wherein the drug resistance signature is compared to genomic data for a human patient to provide patient prognostics related to drug efficacy.

29. The method of claim 20, wherein the drug resistance signature is compared to genomic data for a human patient to anticipate drug toxicity in healthy tissue.

30. The method of claim 20, wherein the drug resistance signature is used to design a therapeutic strategy in which at least one gene from the drug resistance signature is targeted pharmacologically in a human patient, such that drug resistance is prevented or delayed; or such that the drug efficacy increases.
